(19) United States
(12) Patent Application Publication
(10) Pub. No.: US 2013/0034497 A1
(43) Pub. Date: Feb. 7, 2013

(54) IODINE-LABELED HOMOGLUTAMIC ACID AND GLUTAMIC ACID DERIVATIVES

(71) Applicants: Heribert Schmitt-Willich, Berlin (DE); Niels Böhnke, Berlin (DE); Norman Koglin, Berlin (DE); Andre Müller, Berlin (DE); Holger Siebeneicher, Berlin (DE); Matthias Friebe, Berlin (DE)

(72) Inventors: Heribert Schmitt-Willich, Berlin (DE); Niels Böhnke, Berlin (DE); Norman Koglin, Berlin (DE); Andre Müller, Berlin (DE); Holger Siebeneicher, Berlin (DE); Matthias Friebe, Berlin (DE)

(73) Assignee: PIRAMAL IMAGING SA, Matran (CH)

(21) Appl. No.: 13/510,359

(22) PCT Filed: Nov. 15, 2010
(86) PCT No.: PCT/EP10/67500

§ 371 Date: Oct. 24, 2012

(30) Foreign Application Priority Data
Nov. 17, 2009 (EP) 09075506.7

(51) Int. Cl. C07C 229/28; A61K 35/00; A61K 51/04; A61K 31/195; C07F 7/22

(52) U.S. Cl. 424/1.85; 562/444; 514/567; 556/41

(57) ABSTRACT

This invention relates to derivatives of Iodine-labeled homoglutamic acids and glutamic acids and their analogues suitable for labeling or already labeled by Iodine, methods of preparing such compounds, compositions comprising such compounds, kits comprising such compounds or compositions and uses of such compounds, compositions or kits for diagnostic imaging or radiotherapy.

### FIELD OF INVENTION

This invention relates to derivatives of Iodine-labeled homoglutamic acids and glutamic acids and their analogues suitable for labeling or already labeled by Iodine, methods of preparing such compounds, compositions comprising such compounds, kits comprising such compounds or compositions and uses of such compounds, compositions or kits for diagnostic imaging.

### BACKGROUND

The invention relates to the subject matter referred to in the claims i.e. derivatives of Iodine-labeled glutamic or homoglutamic acid and their analogues of the general formulas (I) and (II), their precursors of the formula (III) and to processes for their preparation and their use i.e. in SPECT (Single Photon Emission Computed Tomography)/PET (Positron Emission Tomography) and radiotherapy.

The specific early diagnosis of malignant tumour diseases and their targeted therapy will remain of crucial importance for the survival prognosis of a tumour patient. Regarding diagnosis, non-invasive diagnostic imaging methods are an important aid. In the last years, in particular the PET (Positron Emission Tomography) technology has gained much attention within the diagnostic field. However the preferred radionuclides for PET are ¹⁸F (T_{1/2}=110 min) and ¹¹C (T_{1/2}=20 min): These isotopes have relatively short half-lifes that do not really allow complicated long synthesis routes and purification procedures. Compared to these PET isotopes single photon emitters like ^{99m}Tc (T_{1/2}=6.05 hr) or ¹²³I (T_{1/2}=13.30 hr) have significantly longer half-lives, thus can lead to certain advantages. These include the ability to utilize radiopharmaceuticals that have either slow target uptake or slow background clearance, and the ability to produce the radiopharmaceuticals offsite for distribution to the clinic. In addition, in research a longer half-life makes radiopharmaceutical development more convenient. The simultaneous use of different energy single photon emitters (small animal SPECT imaging or cut and count biodistribution) allows the study of multiple parameters in parallel.

Currently, the use of 2-[¹⁸ F]-fluoro-deoxyglucose (¹⁸F-FDG) in PET is a widely accepted and frequently used auxiliary in the diagnosis and further clinical monitoring of tumour disorders. Malignant tumours compete with the host organism for glucose as nutrient supply (Warburg O., Über den Stoffwechsel der Carcinomzelle [The metabolism of the carcinoma cell], Biochem. Zeitschrift 1924; 152: 309-339; Kell of G., Progress and Promise of FDG-PET Imaging for Cancer Patient Management and Oncologic Drug Development, Clin. Cancer Res. 2005; 11 (8): 2785-2807). Compared to the surrounding cells of the normal tissue, tumour cells usually have an increased glucose metabolism. This is exploited when a labeled glucose derivative which is increasingly transported into the cells, where it is metabolically converted to FDG 6-phosphate via phosphorylation and therefore trapped within the cell (“Warburg effect”). Accordingly, ¹⁸F-labeled FDG is an effective tracer for detecting tumour disorders in patients using the PET technology. Although this method is very sensitive, it has two major limitations, namely an avid accumulation in inflammatory lesions and high uptake in the brain, jeopardizing the diagnosis of brain tumours.

It was shown that the use of radioactive amino acids for SPECT and PET could overcome these shortcomings for the larger part. In the late 80's, several ¹¹C-labelled amino acids like methionine (J. Nucl. Med. 1987, 28, 1037-1040) and tyrosine (Eur. J. Nucl. Med. 1986, 12, 321-324) were used for PET studies. More recently also an emerging amount of ¹⁸F labeled amino acids have been employed for PET imaging (for example (review): Eur. J. Nucl. Med. Mol. Imaging May 2002; 29 (5): 681-90). Some of the ¹⁸F-labeled amino acids are suitable for measuring the rate of protein synthesis but most other derivatives are suitable for measuring the direct cellular uptake in the tumour. Known ¹⁸F-labeled amino acids are derived, for example, from tyrosine amino acids, phenylalanine amino acids, proline amino acids, asparagine amino acids and unnatural amino acids (for example J. Nucl. Med. 1991; 32: 1338-1346, J. Nucl. Med. 1996; 37: 320-325, J. Nucl. Med. 2001; 42: 752-754 and J. Nucl. Med. 1999; 40: 331-338).

In comparison to the PET isotopes ¹¹C and ¹⁸F the introduction of a radioiodine label into an amino acid derivative is more restrictive with regard to in-vivo stability of the incorporated radioiodine isotope. Because of the stronger binding of iodine to an unsaturated carbon atom, the radioiodine labels are attached to vinylic or aromatic sp² carbon centres within the molecule to avoid a fast in vivo deiodination. Therefore in the past only derivatives of aromatic amino acids like tyrosine and phenylalanine have been extensively studied for their use in SPECT imaging and radiotherapy. Amongst others the most prominent examples have been 3-[¹²³I]iodo-α-methyl tyrosine (IMT) (J. Nucl. Med. 1989, 30, 110-112) and p-[¹²³I]iodo-phenylalanine (IPA) (Nucl. Med. Com. 2002, 23, 121-130) for imaging and p-[¹³¹I]iodo-phenylalanine for the treatment of hormone dependent carcinoma (WO2007/060012).

The 3-[¹²³I]iodo-α-methyl tyrosine (IMT) was for example extensively used as a SPECT tracer for brain tumours where the PET tracer ¹⁸F-FDG cannot be employed because of the high background signal in the brain. The uptake of this tracer into tumours occurs mainly by the L-type transport system (Nucl. Med. Comm. 2001, 22, 87-96). The plasma membrane transport system L is the only (efficient) pathway for the import of large branched and aromatic neutral amino acids for many cells. The L-type amino acid transporter 1 (LAT1) is a Na⁺ independent amino acid transporter and is over-expressed in malignant cell as it plays a critical role in cell growth and proliferation. For functional expression LAT1 requires the heavy chain of the surface antigen 4F2 (heavy chain 4F2hc). The increased accumulation is mainly determined by strongly increased amino acid transport activity rather than incorporation into proteins. However, a major drawback limiting the applicability of this tracer is the high renal accumulation (Nucl. Med. Comm. 2002, 23, 121-130). Despite the unfavorable biodistribution the tyrosine example clearly shows that the employment of labeled amino acids as tumour tracers can show higher tumor specificity then the current “Goldstandard” ¹⁸F-FDG.

The FDG has another major disadvantage. As it is preferably accumulated in cells having an elevated glucose metabolism, it can also, under different pathological and physiological conditions, be taken up by cells and tissues involved at infection sites or areas of wound healing (summarized in J. Nucl. Med. Technol. (2005), 33, 145-155). Frequently, it is still difficult to ascertain whether a lesion detected via FDG-PET is really of neoplastic origin or is the result of other physiological or pathological conditions of the tissue. Overall, the diagnosis by FDG-PET in oncology has a sensitivity of 84% and a specificity of 88% (Gambhir et al., “A tabulated summary of the FDG PET literature”, J. Nucl. Med. 2001, 42, 1-93S).

Similarly to glucose glutamic acid and glutamine also show an increased metabolism in proliferating tumour cells (Medina, J. Nutr. 1131: 2539S-2542S, 2001; Souba, Ann Surg 218: 715-728, 1993). The increased rate of protein and nucleic acid synthesis and the energy generation per se are thought to be the reasons for the increased glutamine consumption in tumour cells. The synthesis of corresponding C-11- and C-14-labelled compounds, which are thus identical to the natural substrate, has already been described in the literature (for example Antoni, Enzyme Catalyzed Synthesis of L-[4-C-11]aspartate and L-[5-C-11]glutamate. J. Labelled Compd. Radiopharm. 44; (4) 2001: 287-294 and Buchanan, The biosynthesis of showdomycin: studies with stable isotopes and the determination of principal precursors, J. Chem. Soc. Chem. Commun.; EN; 22; 1984; 1515-1517). First tests with the C-11-labeled compound indicate no significant accumulation in tumors.

Radiotherapy in the clinical practice commonly makes use of ¹³¹I-sodium iodide to treat hypothyroidism and dedifferentiated thyroid carcinoma, based on the physiological accumulation if iodine in the thyroid. Targeted radiotherapy requires a molecule which has a specificity for tumor tissue coupled to a radionuclide with the appropriate physical characteristics (Perkins A C, In vivo molecular targeted radiotherapy Biomed Imaging Interv J 2005; 1 (2):e9). This combination results in selective irradiation of the tumor cells with relative sparing of normal tissues. One example in this area is the catecholamine analogue [¹³¹I]MIBG, used in the clinic to treat neuroblastoma.

It is an object of the present invention to provide novel compounds which, in radioiodine-labeled form, are suitable for diagnosis and/or radiotherapy.

This object is achieved by the provision according to the invention of radioiodine-labeled glutamic acid and homoglutamic acid derivatives of the general formula (I) and (II), including single isomers, enantiomers, diastereomers, tautomers, E- and Z-isomers, mixtures thereof, and suitable salts thereof.

### SUMMARY

The invention relates to the subject matter referred to in the claims i.e. derivatives of iodinated glutamic or homoglutamic acid and their analogues of the general formulas (I) and (II), their precursors of the formula (III) and to processes for their preparation and their use i.e. in SPECT (Single Photon Emission Computed Tomography)/PET (Positron Emission Tomography) and radiotherapy.

### FIGURES

FIG. 1: Concentration dependent blocking of 3H-Glutamic acid uptake in H460 cells using different concentrations of (2S,4S)-2-Amino-4-(3-[4-iodophenoxy]propyl)-pentanedioic acid.

FIG. 2: Examination of biological activity of (2S,4S)-2-Amino-4-(3-[4-[I-125]-iodophenoxy]-propylypentanedioic acid in a tumor cell uptake/binding experiment. (NCl-H460 cells, up to 30 min incubation with I125-labeled derivative).

FIG. 3: Examination of biological activity of (2S,4S)-2-Amino-4-(3-[4-[I-125]-iodophenoxy]propylypentanedioic acid in a cell competition experiment. (NCl-H460 cells, 30 min incubation with I125-labeled derivative in PBS-buffer, concentration of “cold” derivative 1 mM).

FIG. 4: Examination of biological activity of (2S,4S)-2-Amino-4-(4-iodo-benzyl)-pentanedioic acid in a cell competition experiment. (NCl-H460 cells, A549 cells, 10 min incubation with 1 μCi 3H-Glutamic acid in PBS-buffer, concentration of test compound 1 mM).

FIG. 5: Determination of biological activity of (2S,4S)-2-Amino-4-(4-hydroxy-3-[I-125]-iodobenzyl)-pentanedioic acid in a cell competition experiment. (NCl-H460 cells, 10 min incubation with [I125]-labeled derivative in PBS-buffer, concentration of L-Glutamate 1 mM).

FIG. 6 The time dependence of uptake of (2S,4S)-2-Amino-4-(4-[I-125]-iodo-benzyl)-pentanedioic acid was determined. H460 cells were incubated with 0.25 MBq (2S,4S)-2-Amino-4-(4-[I-125]-iodo-benzyl)-pentanedioic acid for up to 60 min and the cell-bound fraction was determined after 10, 20, 30 and 60 min).

FIG. 7 Examination of retention of (2S,4S)-2-Amino-4-(4-[I-125]odo-benzyl)-pentanedioic acid in H460 tumor cells. H460 cells were loaded with 0.25 MBq (2S,4S)-2-Amino-4-(4-[I-125]-iodo-benzyl)-pentanedioic acid for 30 min in PBS/BSA. After washing, the cells were incubated with new buffer (without radioactivity) for additional 10, 20, 30 min. The release of radioactivity into the supernatant as well as the retention inside the cells was determined.

FIG. 8 SPECT imaging with (2S,4S)-2-Amino-4-(4-[I-125]odo-benzyl)-pentanedioic acid after injection into H460 tumor bearing mouse.

FIG. 9 Examination of biological activity of (S)-2-Amino-5-(4-iodobenzyl)-hexanedioic acid in a cell-competition-experiment (H460 cells, 30 min incubation with 3H-glutamic acid in PBS-Puffer, concentration of competitor 1 mM and 0.1 mM).

### DESCRIPTION

In a first aspect, the invention is directed to compounds of the general formula (I)

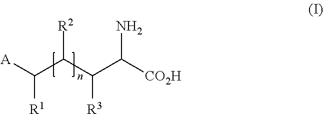

wherein
n=0 or 1;
A is selected from the group comprising

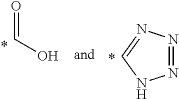

wherein * indicates the atom of connection of A;
R¹, R² and R³ are independently from each other selected from Hydrogen and X with the proviso that one of R¹ , R² and R³ is X,
wherein X is
Iodo-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
Iodo-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S. and wherein the heteroaryl moiety is optionally substituted by a methyl group
or
Iodo-CH═CH—(CH₂)ₘ, wherein m=1-3.

Formula (I) encompasses single isomers, diastereomers, tautomers, E- and Z-isomers, enantiomers, mixtures thereof, and suitable salts thereof.

Preferably, the Iodine is ¹²³I, ¹²⁴I or ¹²⁵I.

Preferably, the Iodine is ¹²⁷I. More preferably, when Iodine is ¹²⁷I then compound of formula I is never (2R,4S)-2-Amino-4-(m-iodo)benzyl pentanedioic acid or (2R,4S)-2-Amino-4-(p-iodo)benzyl pentanedioic acid.

Preferably, the Iodine is ¹³¹I.

Preferably, A is a carboxylic group.

Preferably, R² and R³ are Hydrogen and R¹ is X.

Preferably, X is
Iodo-aryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
or
Iodo-CH═CH—(CH₂)ₘ, wherein m=1-3.

Preferably, branched or straight C₁-C₅ alkyl is C₁-C₃ alkyl, C₁ alkyl (CH₂), C₂ alkyl ((CH₂)₂), C₃ alkyl (e.g. (CH₂)₃), C₄ alkyl (e.g. (CH₂)₄), or C₅ alkyl (e.g. (CH₂)₅)

More preferably, the alkyl chain is C₁-C₃ alkyl.

Preferably, aryl is phenyl or naphthyl groups e.g. 1-naphthyl and 2-naphthyl, more preferably phenyl.

Preferably, heteroaryl is thienyl, furanyl, pyrrolyl, oxazolyl, thiazolyl, imidazolyl, pyrazolyl, pyridinyl, pyrazinyl or pyrimidinyl, more preferably pyridinyl.

Preferably, m is 1 or 2. Preferably, m is 3.

Preferably, n is 0. Preferably, n is 1.

More preferably, the compound of formula I is never 2-Amino-4-(m-iodo)benzyl pentanedioic acid , 2-Amino-4-(p-iodo)benzyl pentanedioic acid, (2R,4S)-2-Amino-4-(m-iodo)benzyl pentanedioic acid or (2R,4S)-2-Amino-4-(p-iodo)benzyl pentanedioic acid. Even more preferably, the compound of formula I is never (2R,4S)-2-Amino-4-(m-iodo)benzyl pentanedioic acid or (2R,4S)-2-Amino-4-(p-iodo)benzyl pentanedioic acid.

Preferably, A is

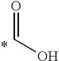

and

X is Iodo-aryl-G-CH₂ is Iodo-phenyl-G-CH₂ wherein G is C₁-C₃-alkyl or —O-C₁-C₃-alkyl and wherein aryl is optionally substituted with OH. More preferably, Iodo-phenyl-C₁-C₃-alkyl-CH₂ or Iodo-phenyl-O-C₁-C₃-alkyl-CH₂.

Preferably, A is

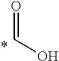

and

X is Iodo-heteroaryl-G-CH₂ is Iodo-pyridinyl-G-CH2 or Iodo-thienyl-G-CH2 wherein G is C₁-C₃-alkyl or —C(O)—NH-C₁-C₃-alkyl.

Preferably, A is

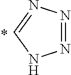

and

X is Iodo-aryl-G-CH₂ is Iodo-phenyl-G-CH₂ wherein G is C₁-C₃-alkyl or —O—C₁-C₃-alkyl and wherein aryl is optionally substituted with OH. More preferably, Iodo-phenyl-C₁-C₃-alkyl-CH₂ or Iodo-phenyl-O-C₁-C₃-alkyl-CH₂.

Preferably, A is

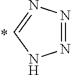

and

X is Iodo-heteroaryl-G-CH₂ is Iodo-pyridinyl-G-CH2 or Iodo-thienyl-G-CH2 wherein G is C₁-C₃-alkyl or —C(O)—NH-C₁-C₃-alkyl.

In a first embodiment, the invention is directed to a compound of general formula (I) wherein

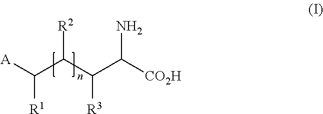

wherein
n=1;
A is selected from the group comprising

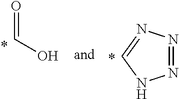

wherein * indicates the atom of connection of A;
R¹, R² and R³ are independently from each other selected from Hydrogen and X with the proviso that one of R¹ , R² and R³ is X,
wherein X is
Iodo-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
Iodo-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S. and wherein the heteroaryl moiety is optionally substituted by a methyl group
or
Iodo-CH═CH—(CH₂)ₘ, wherein m=1-3.

Preferably, compound of general formula (I) wherein n=1 is a compound of general formula (I-H2S)

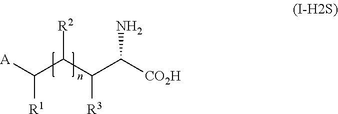

wherein R¹ to R³, A and X are disclosed above.

The preferred features R¹ to R³, A and X disclosed for compound of general formula (I) above are incorporated herein.

In a second embodiment, the invention is directed to a compound of general formula (I) wherein

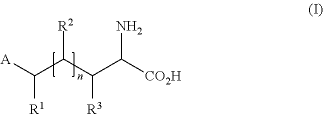

wherein
n=0;
A is selected from the group comprising

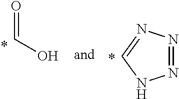

wherein * indicates the atom of connection of A;
R¹, R² and R³ are independently from each other selected from Hydrogen and X with the proviso that one of R¹ , R² and R³ is X,
wherein X is
Iodo-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
Iodo-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S. and wherein the heteroaryl moiety is optionally substituted by a methyl group
or
Iodo-CH═CH—(CH₂)ₘ, wherein m=1-3.

Preferably, compound of general formula (I) wherein n=0 is a compound of general formula (I-G2S)

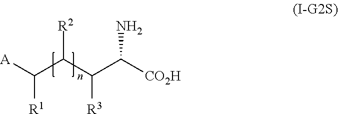

wherein R¹ to R³ , A and X are disclosed above.

The preferred features R¹ to R³ , A and X disclosed for compound of general formula (I) above are incorporated herein.

Embodiments and preferred features can be combined together and are within the scope of the invention.

Invention compounds are selected from but not limited to (2S,4S)-2-Amino-4-(4-hydroxy-3-iodo-benzyl)-pentanedioic acid

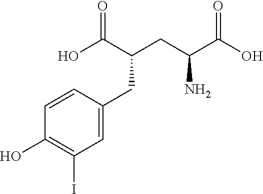 [SMILES: N[C@@H](C[C@H](Cc1ccc(O)c(I)c1)C(=O)O)C(=O)O]

(2S,4S)-2-Amino-4-(4-hydroxy-3-[125-I]iodo-benzyl)-pentanedioic acid

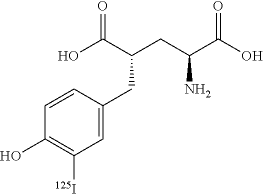 [SMILES: N[C@@H](C[C@H](Cc1ccc(O)c([125I])c1)C(=O)O)C(=O)O]

(2S,4S)-2-Amino-4-[3-(4-iodo-phenoxy)-propyl]-pentanedioic acid

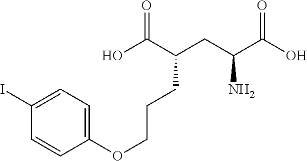 [SMILES: N[C@@H](C[C@H](CCCOc1ccc(I)cc1)C(=O)O)C(=O)O]

(2S,4S)-2-Amino-4-[3-(4-125-I]iodo-phenoxy)-propyl-pentanedioic acid

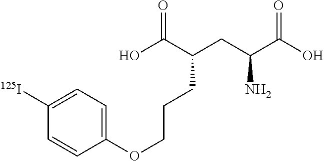 [SMILES: N[C@@H](C[C@H](CCCOc1ccc([125I])cc1)C(=O)O)C(=O)O]

(S)-2-Amino-7-(4-iodo-phenoxy)-4-(1H-tetrazol-5-yl)-heptanoic acid

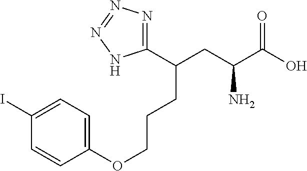 [SMILES: N[C@@H](CC(CCCOc1ccc(I)cc1)c1nnn[nH]1)C(=O)O]

(S)-2-Amino-7-(4-[125-I]iodo-phenoxy)-4-(1H-tetrazol-5-yl)-heptanoic acid

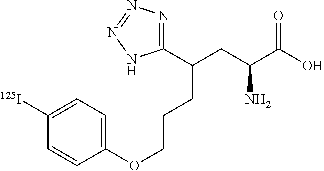 [SMILES: N[C@@H](CC(CCCOc1ccc([125I])cc1)c1nnn[nH]1)C(=O)O]

(2S,4S)-2-Amino-4-(4-iodo-benzyl)-pentanedioic acid

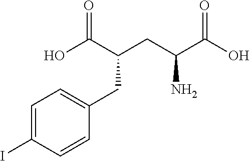 [SMILES: N[C@@H](C[C@H](Cc1ccc(I)cc1)C(=O)O)C(=O)O]

(2S,4S)-2-Amino-4-(4-[125-I]iodo-benzyl)-pentanedioic acid

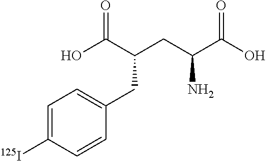 [SMILES: N[C@@H](C[C@H](Cc1ccc([125I])cc1)C(=O)O)C(=O)O]

(S)-2-Amino-4-(2-iodo-thiophen-3-ylmethyl)-pentanedioic acid

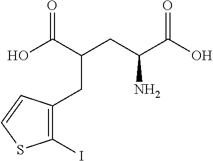 [SMILES: N[C@@H](CC(Cc1ccsc1I)C(=O)O)C(=O)O]

(S)-2-Amino-4-(2-[125-I]iodo-thiophen-3-ylmethyl)-pentanedioic acid

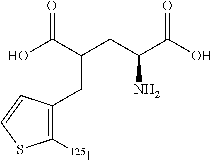 [SMILES: N[C@@H](CC(Cc1ccsc1[125I])C(=O)O)C(=O)O]

(2S,4S)-2-Amino-4-{3-[(2-iodo-pyridine-4-carbonyl)-amino]-propyl}-pentanedioic acid

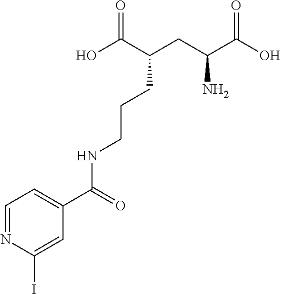 [SMILES: N[C@@H](C[C@H](CCCNC(=O)c1ccnc(I)c1)C(=O)O)C(=O)O]

(2S,4S)-2-Amino-4-{3-[(2-[125-I]iodo-pyridine-4-carbonyl)-amino]-propyl}-pentanedioic acid

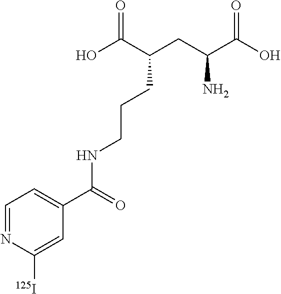 [SMILES: N[C@@H](C[C@H](CCCNC(=O)c1ccnc([125I])c1)C(=O)O)C(=O)O]

(2S,4S)-2-Amino-4-[3-(3-iodo-benzoylamino)-propyl]-pentanedioic acid

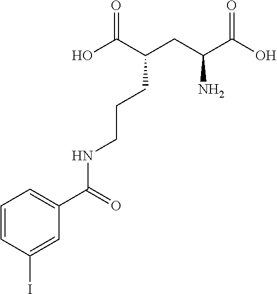 [SMILES: N[C@@H](C[C@H](CCCNC(=O)c1cccc(I)c1)C(=O)O)C(=O)O]

(2S,4S)-2-Amino-4-[3-(3-[125-I]iodo-benzoylamino)-propyl]-pentanedioic acid

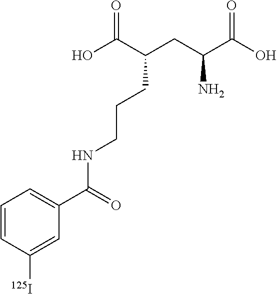 [SMILES: N[C@@H](C[C@H](CCCNC(=O)c1cccc([125I])c1)C(=O)O)C(=O)O]

(S)-2-Amino-5-(4-iodo-phenyl)-4-(1H-tetrazol-5-yl)-pentanoic acid

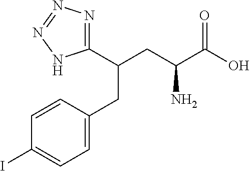 [SMILES: N[C@@H](CC(Cc1ccc(I)cc1)c1nnn[nH]1)C(=O)O]

(S)-2-Amino-5-(4-[125-I]iodo-phenyl)-4-(1H-tetrazol-5-yl)-pentanoic acid

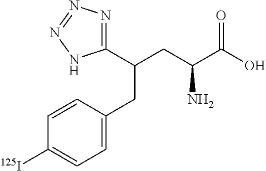 [SMILES: N[C@@H](CC(Cc1ccc([125I])cc1)c1nnn[nH]1)C(=O)O]

(2S,5S)-2-Amino-5-(4-iodo-benzyl)-hexanedioic acid

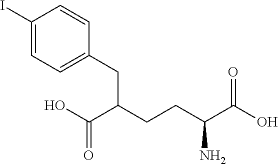 [SMILES: N[C@@H](CCC(Cc1ccc(I)cc1)C(=O)O)C(=O)O]

and

(S)-2-Amino-5-(4-iodobenzyl)-hexanedioic acid

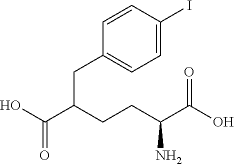 [SMILES: N[C@@H](CCC(Cc1ccc(I)cc1)C(=O)O)C(=O)O]

In a second aspect, the invention is directed to compounds of the general formula (II)

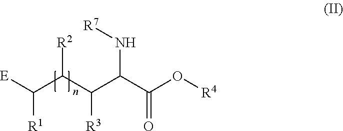

wherein
n=0 or 1;
E is selected from the group comprising

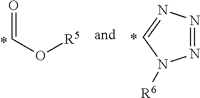

wherein * indicates the atom of connection of E;
R¹, R² and R³ are independently from each other selected from Hydrogen and X with the proviso that one of R¹ , R² and R³ is X,
wherein X is
Iodo-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
Iodo-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S and wherein the heteroaryl moiety is optionally substituted by a methyl group
or
Iodo-CH═CH—(CH₂)ₘ, wherein m=1-3;
R⁴=Hydrogen or O-protecting group;
R⁵=Hydrogen or O-protecting group;
R⁶=Hydrogen or triphenylmethyl;
R⁷=Hydrogen or N-protecting group;
with the proviso, that at least one of the substituents R⁴, R⁵, R⁶ or R⁷ is not Hydrogen.
Formula (II) encompasses single isomers, diastereomers, tautomers, E- and Z-isomers, enantiomers, mixtures thereof, and suitable salts thereof.

Preferably, the Iodine is ¹²³I, ¹²⁴I or ¹²⁵I.

Preferably, the Iodine is ¹²⁷I.

Preferably, the Iodine is ¹³¹I.

Preferably, E is

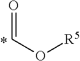

wherein * indicates the atom of connection of E.

Preferably, R² and R³ are Hydrogen and R¹ is X.

The compounds of formula II are Iodine-labeled compounds wherein the functional group(s) such as OH and NH₂ all or in part are protected with suitable protecting group(s) defined as R⁴ to R⁷, respectively.

The preferred features n, R¹ to R³ disclosed for compound of general formula (I) are incorporated herein.

O-protecting group is selected from the group comprising

Methyl, Ethyl, Propyl, Butyl and t-Butyl. Preferably, O-protecting group is selected from the group comprising Methyl, Ethyl and t-Butyl. More preferably, O-protecting group is t-Butyl. Preferably, R⁴ and R⁵ are O-protecting groups.

N-protecting group is selected from the group comprising

Carbobenzyloxy (Cbz), tert-Butyloxycarbonyl (BOC), 9-Fluorenylmethyloxycarbonyl (FMOC), and Triphenylmethyl. Preferably, N-protecting group is selected from the group comprising Carbobenzyloxy (Cbz), tert-Butyloxycarbonyl (BOC) and 9-Fluorenylmethyloxycarbonyl (FMOC). More preferably, N-protecting group is tert-Butyloxycarbonyl (BOC) or 9-Fluorenylmethyloxycarbonyl (FMOC).

Preferably, R⁷ is a N-protecting group.

Preferably, aryl is phenyl or naphthyl groups e.g. 1-naphthyl and 2-naphthyl.

Preferably, heteroaryl is thienyl, furanyl, pyrrolyl, oxazolyl, thiazolyl, imidazolyl, pyrazolyl, pyridinyl, pyrazinyl or pyrimidinyl.

Preferably, m is 1 or 2. Preferably, m is 3.

Preferably, n is 0. Preferably, n is 1.

Preferably, E is

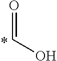

and

X is Iodo-aryl-G-CH₂ is Iodo-phenyl-G-CH₂ wherein G is C₁-C₃-alkyl or —O-C₁-C₃-alkyl and wherein aryl is optionally substituted with OH. More preferably, Iodo-phenyl-C₁-C₃-alkyl-CH₂ or Iodo-phenyl-O-C₁-C₃-alkyl-CH₂.

Preferably, E is

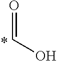

and

X is Iodo-heteroaryl-G-CH₂ is Iodo-pyridinyl-G-CH2 or Iodo-thienyl-G-CH2 wherein G is C₁-C₃-alkyl or —C(O)—NH-C₁-C₃-alkyl.

Preferably. E is

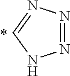

and

X is Iodo-aryl-G-CH₂ is Iodo-phenyl-G-CH₂ wherein G is C₁-C₃-alkyl or —O-C₁-C₃-alkyl and wherein aryl is optionally substituted with OH. More preferably, Iodo-phenyl-C₁-C₃-alkyl-CH₂ or Iodo-phenyl-O-C₁-C₃-alkyl-CH₂.

Preferably, E is

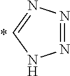

and

X is Iodo-heteroaryl-G-CH₂ is Iodo-pyridinyl-G-CH2 or Iodo-thienyl-G-CH2 wherein G is C₁-C3-alkyl or —C(O)—NH-C₁-C₃-alkyl.

Preferably, E is

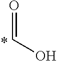

and
R⁴ is t-Butyl;
R⁵ is t-Butyl; and
R⁷ is tert-Butoxycarbonyl (BOC).

In a first embodiment, the invention is directed to a compound of general formula (II) wherein

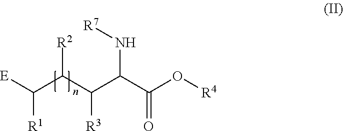

wherein
n=1;
E is selected from the group comprising

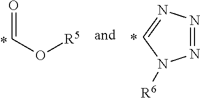

wherein * indicates the atom of connection of E;
R¹, R² and R³ are independently from each other selected from Hydrogen and X with the proviso that one of R¹ , R² and R³ is X,
wherein X is
Iodo-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
Iodo-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S and wherein the heteroaryl moiety is optionally substituted by a methyl group
or
Iodo-CH═CH—(CH₂)ₘ, wherein m=1-3;
R⁴=Hydrogen or O-protecting group;
R⁵=Hydrogen or O-protecting group;
R⁶=Hydrogen or triphenylmethyl;
R⁷=Hydrogen or N-protecting group;
with the proviso, that at least one of the substituents R⁴, R⁵, R⁶ or R⁷ is not Hydrogen.

Preferably, compound of general formula (II) wherein n=1 is a compound of general formula (II-H2S)

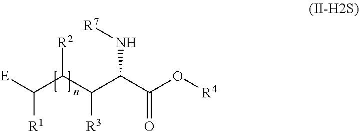

wherein R¹ , R², R³ , R⁴, R⁷, E and X are disclosed above.

The preferred features R¹ , R², R³ , R⁴, R⁷, E and X disclosed above for compound of general formula (II) above are incorporated herein.

In a second embodiment, the invention is directed to a compound of general formula (II) wherein

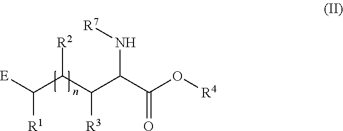

wherein
n=0;
E is selected from the group comprising

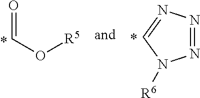

wherein * indicates the atom of connection of E;
R¹, R² and R³ are independently from each other selected from Hydrogen and X with the proviso that one of R¹ , R² and R³ is X,
wherein X is
Iodo-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
Iodo-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S and wherein the heteroaryl moiety is optionally substituted by a methyl group
or
Iodo-CH═CH—(CH₂)ₘ, wherein m=1-3;
R⁴=Hydrogen or O-protecting group;
R⁵=Hydrogen or O-protecting group;
R⁶=Hydrogen or triphenylmethyl;
R⁷=Hydrogen or N-protecting group;
with the proviso, that at least one of the substituents R⁴, R⁵, R⁶or R⁷ is not Hydrogen.

Preferably, compound of general formula (I) wherein n=0 is a compound of general formula (II-G2S)

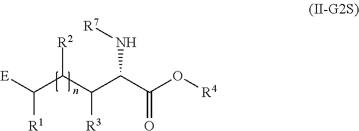

wherein R¹ , R², R³ , R⁴, R⁷, E and X are disclosed above.

The preferred features R¹ , R², R³ , R⁴, R⁷, E and X disclosed above for compound of general formula (II) above are incorporated herein.

The preferred features disclosed for compound of general formula (I) are herein incorporated.

Invention compounds are selected from but not limited to

(2S,4S)-2-tert-Butoxycarbonylamino-4-[3-(4-iodo-phenoxy)-propyl]-pentanedioic acid di-tert-butyl ester

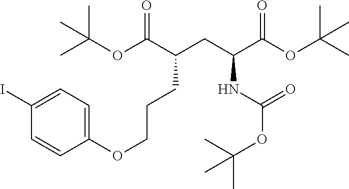 [SMILES: CC(C)(C)OC(=O)N[C@@H](C[C@H](CCCOc1ccc(I)cc1)C(=O)OC(C)(C)C)C(=O)OC(C)(C)C]

(2S,4S)-2-tert-Butoxycarbonylamino-4-(4-[125-I]iodo-benzyl)-pentanedioic acid di-tert-butyl ester

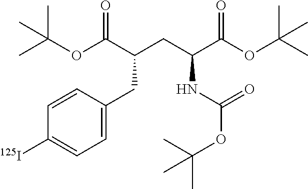 [SMILES: CC(C)(C)OC(=O)N[C@@H](C[C@H](Cc1ccc([125I])cc1)C(=O)OC(C)(C)C)C(=O)OC(C)(C)C]

(2S,4S)-2-tert-Butoxycarbonylamino-4-{3-[(2-[125-I]iodo-pyridine-4-carbonyl)-amino]propyl}-pentanedioic acid di-tert-butyl ester

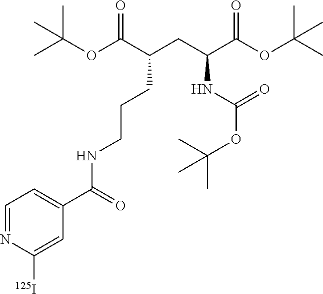 [SMILES: CC(C)(C)OC(=O)N[C@@H](C[C@H](CCCNC(=O)c1ccnc([125I])c1)C(=O)OC(C)(C)C)C(=O)OC(C)(C)C]

(2S,4S)-2-tert-Butoxycarbonylamino-4-[3-(3-[125-I]iodo-benzoylamino)-propyl]-pentanedioic acid di-tert-butyl ester

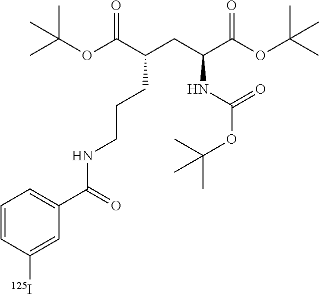 [SMILES: CC(C)(C)OC(=O)N[C@@H](C[C@H](CCCNC(=O)c1cccc([125I])c1)C(=O)OC(C)(C)C)C(=O)OC(C)(C)C]

(2S,4S)-2-tert-Butoxycarbonylamino-4-(3-iodo-allyl)-pentanedioic acid di-tert-butyl ester

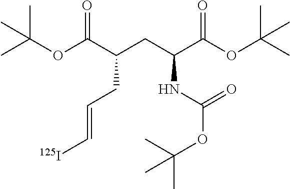 [SMILES: CC(C)(C)OC(=O)N[C@@H](C[C@H](C/C=C/[125I])C(=O)OC(C)(C)C)C(=O)OC(C)(C)C]

In a third aspect, the invention is directed to compounds of the general formula (III)

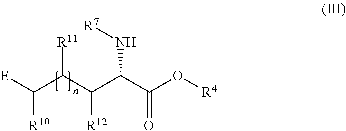

wherein
n=0 or 1;
E is selected from the group comprising

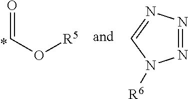

wherein * indicates the atom of connection of E;
R¹⁰, R¹¹ and R¹² are independently from each other selected from Hydrogen and Y with the proviso that one of R^{10, R}¹¹ and R¹² is Y,
wherein Y is

L-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
L-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S and wherein the heteroaryl moiety is optionally substituted by a methyl group
or
L-CH═CH—(CH₂)ₘ, wherein m=1-3
wherein L is
(R¹³)₃Sn, (R¹³)₃Si or (HO)₂B,
wherein R¹³ is C₁-C₄ Alkyl, preferably n-Butyl;
R⁴=Hydrogen or O-protecting group;
R⁵=Hydrogen or O-protecting group;
R⁶=Hydrogen or triphenylmethyl;
R⁷=Hydrogen or N-protecting group.

Formula (III) encompasses single isomers, diastereomers, tautomers, E- and Z-isomers, enantiomers, mixtures thereof, and suitable salts thereof. The compounds of formula III are compounds suitable for coupling iodine wherein the functional group(s) such as OH, NH and NH₂ are protected with suitable protecting group(s) such as R⁴, R⁵, R⁶ and R⁷, respectively.

Preferably, E is

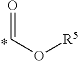

wherein * indicates the atom of connection of E.

Preferably, R¹¹ and R¹² are Hydrogen and R¹⁰ is Y.

O-protecting group is selected from the group comprising

Methyl, Ethyl, Propyl, Butyl and t-Butyl. Preferably, O-protecting group is selected from the group comprising Methyl, Ethyl and t-Butyl. More preferably, O-protecting group is t-Butyl. Preferably, R⁴ and R⁵ are O-protecting groups.

N-protecting group is selected from the group comprising

Carbobenzyloxy (Cbz), tert-Butyloxycarbonyl (BOC), 9-Fluorenylmethyloxycarbonyl (FMOC), and Triphenylmethyl. Preferably, N-protecting group is selected from the group comprising Carbobenzyloxy (Cbz), tert-Butyloxycarbonyl (BOC) and 9-Fluorenylmethyloxycarbonyl (FMOC). More preferably, N-protecting group is tert-Butyloxycarbonyl (BOC) or 9-Fluorenylmethyloxycarbonyl (FMOC). Preferably, R⁷ is a N-protecting group.

Preferably, aryl is phenyl or naphthyl groups e.g. 1-naphthyl and 2-naphthyl.

Preferably, heteroaryl is thienyl, furanyl, pyrrolyl, oxazolyl, thiazolyl, imidazolyl, pyrazolyl, pyridinyl, pyrazinyl or pyrimidinyl.

Preferably, m is 1 or 2. Preferably, m is 3.

Preferably, n is 0. Preferably, n is 1.

Preferably, E is

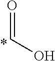

and

Y is L-aryl-G-CH₂ is L-phenyl-G-CH₂ wherein G is C₁-C₃-alkyl or —O-C₁-C₃-alkyl and wherein aryl is optionally substituted with OH and L is (R¹³)₃Sn—, or (R¹³)₃Si—. More preferably, L-phenyl-C₁-C₃-alkyl-CH₂ or L-phenyl-O-C₁-C₃-alkyl-CH₂ wherein L is (R¹³)₃Sn— and R¹³ is n-butyl.

Preferably, E is

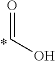

and

Y is L-heteroaryl-G-CH₂ is L-pyridinyl-G-CH2 or L-thienyl-G-CH2 wherein G is C₁-C₃-alkyl or —C(O)—NH-C₁-C₃-alkyl and L is (R¹³)₃Sn—, or (R¹³)₃Si— wherein L is (R¹³)₃Sn— and R¹³ is n-butyl.

Preferably, E is

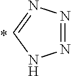

and

Y is L-aryl-G-CH₂ is L-phenyl-G-CH₂ wherein G is C₁-C₃-alkyl or —O-C₁-C₃-alkyl and wherein aryl is optionally substituted with OH and L is (R¹³)₃Sn—, or (R¹³)₃Si—. More preferably, L-phenyl-C₁-C₃-alkyl-CH₂ or L-phenyl-O-C₁-C₃-alkyl-CH₂ wherein L is (R¹³)₃Sn— and R¹³ is n-butyl.

Preferably, E is

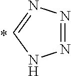

and

Y is L-heteroaryl-G-CH₂ is L-pyridinyl-G-CH2 or L-thienyl-G-CH2 wherein G is C₁-C₃-alkyl or —C(O)—NH-C₁-C₃-alkyl and L is (R¹³)₃Sn—, or (R¹³)₃Si— wherein L is (R¹³)₃Sn— and R¹³ is n-butyl.

Preferably, E is

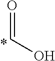

and
R⁴ is t-Butyl;
R⁵ is t-Butyl; and
R⁷ is tert-Butoxycarbonyl (BOC).

In a first embodiment, the invention is directed to a compound of general formula (III)

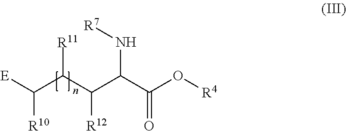

wherein
n=1;
E is selected from the group comprising

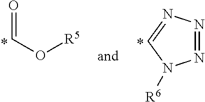

wherein * indicates the atom of connection of E;
R¹⁰, R¹¹ and R¹² are independently from each other selected from Hydrogen and Y with the proviso that one of R¹⁰, R¹¹ and R¹² is Y,
wherein Y is
L-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
L-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S and wherein the heteroaryl moiety is optionally substituted by a methyl group
or
L-CH═CH—(CH₂)ₘ, wherein m=1-3
wherein L is
(R¹³)₃Sn, (R¹³)₃Si or (HO)₂B,
wherein R¹³ is C₁-C₄ Alkyl, preferably n-Butyl;
R⁴=Hydrogen or O-protecting group;
R⁵=Hydrogen or O-protecting group;
R⁶=Hydrogen or triphenylmethyl;
R⁷=Hydrogen or N-protecting group.

Preferably, compound of general formula (III) wherein n=1 is a compound of general formula (III-H2S)

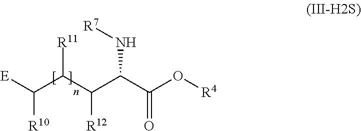

wherein R¹⁰ , R¹¹, R¹², R⁴, R⁵, R⁶, R⁷, E and Y are disclosed above.

The preferred features R¹⁰ , R¹¹, R¹², R⁴, R⁵, R⁶, R⁷, E and Y disclosed above for compound of general formula (III) above are incorporated herein.

In a second embodiment, the invention is directed to a compound of general formula (III)

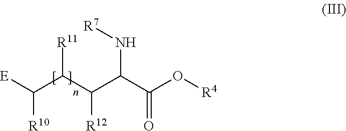

wherein
n=0;
E is selected from the group comprising

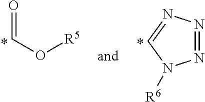

wherein * indicates the atom of connection of E;
R¹⁰, R¹¹ and R¹² are independently from each other selected from Hydrogen and Y with the proviso that one of R¹⁰, R¹¹ and R¹² is Y,
wherein Y is

L-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
L-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S and wherein the heteroaryl moiety is optionally substituted by a methyl group
or
L-CH═CH—(CH₂)ₘ, wherein m=1-3
wherein L is
(R¹³)₃Sn, (R¹³)₃Si or (HO)₂B,
wherein R¹³ is C₁-C₄ Alkyl, preferably n-Butyl;
R⁴=Hydrogen or O-protecting group;
R⁵=Hydrogen or O-protecting group;
R⁶=Hydrogen or triphenylmethyl;
R⁷=Hydrogen or N-protecting group.

Preferably, compound of general formula (III) wherein n=0 is a compound of general formula (III-G2S)

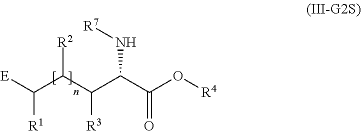

wherein R¹ , R², R³ , R⁴, R⁵, R⁶ , R⁷, E and Y are disclosed above.

The preferred features R¹ , R², R³ , R⁴, R⁷, E and Y disclosed above for compound of general formula (II) above are incorporated herein.

Embodiments and preferred features can be combined together and are within the scope of the invention. The preferred features disclosed for compound of general formula (I) or (II) are incorporated herein.

Invention compounds are selected from but not limited to

(2S,4S)-2-tert-Butoxycarbonylamino-4-(4-tributylstannanyl-benzyl)-pentanedioic acid di-tert-butyl ester

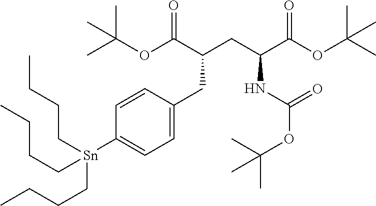 [SMILES: CCC[CH2][Sn]([CH2]CCC)([CH2]CCC)[c]1ccc(C[C@@H](C[C@H](NC(=O)OC(C)(C)C)C(=O)OC(C)(C)C)C(=O)OC(C)(C)C)cc1]

(2S,4S)-2-tert-Butoxycarbonylamino-4-[3-(4-tributylstannanyl-phenoxy)-propyl]-pentanedioic acid di-tert-butyl ester

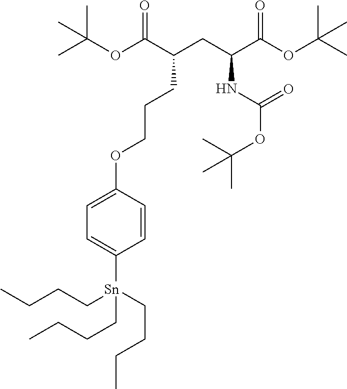 [SMILES: CCC[CH2][Sn]([CH2]CCC)([CH2]CCC)[c]1ccc(OCCC[C@@H](C[C@H](NC(=O)OC(C)(C)C)C(=O)OC(C)(C)C)C(=O)OC(C)(C)C)cc1]

(2S,4S)-2-tert-Butoxycarbonylamino-4-[3-(3-tributylstannanyl-benzoylamino)-propyl]-pentanedioic acid di-tert-butyl ester

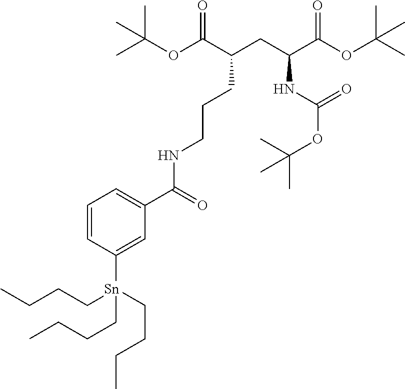 [SMILES: CCC[CH2][Sn]([CH2]CCC)([CH2]CCC)[c]1cccc(C(=O)NCCC[C@@H](C[C@H](NC(=O)OC(C)(C)C)C(=O)OC(C)(C)C)C(=O)OC(C)(C)C)c1]

di-tert-butyl (4S)-N-(tert-butoxycarbonyl)-4-[(2E)-3-(dihydroxyboryl)prop-2-en-1-yl]-L-glutamate

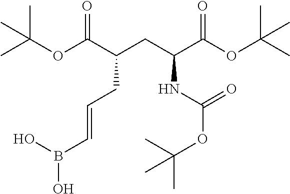 [SMILES: CC(C)(C)OC(=O)N[C@@H](C[C@H](C/C=C/B(O)O)C(=O)OC(C)(C)C)C(=O)OC(C)(C)C]

In a fourth aspect, the invention is directed to a composition comprising compounds of the general formula (I), (II), (III), or mixture thereof and pharmaceutically acceptable carrier or diluent.

The person skilled in the art is familiar with auxiliaries, vehicles, excipients, diluents, carriers or adjuvants which are suitable for the desired pharmaceutical formulations, preparations or compositions on account of his/her expert knowledge.

The administration of the compounds, pharmaceutical compositions or combinations according to the invention is performed in any of the generally accepted modes of administration available in the art. Intravenous deliveries are preferred.

Generally, the compositions according to the invention is administered such that the dose of the active compound for imaging is in the range of 37 MBq (1 mCi) to 740 MBq (20 mCi). In particular, a dose in the range from 150 MBq to 370 MBq will be used.

There preferred dose of the radiolabeled compound for radiotherapeutic purposes is in the range of 1850 MBq (50 mCi) to 11100 MBq (300 mCi) depending on dose limiting organ and body weight.

In a fifth aspect, the invention is directed to a method for obtaining compounds of formula (I), (II) or mixtures thereof.

The method of the invention is an iodine-labeling method.

Preferably, the iodine-labeling method concerns a method for labeling invention compounds with Iodine containing moiety wherein the Iodine containing moiety preferably comprises ¹²³I, ¹²⁴I, ¹²⁵I, ¹²⁷I or ¹³¹I.

More preferably, Iodine containing moiety comprises ¹²³I, ¹²⁴I, ¹²⁵I or ¹³¹I.

Preferably, the Iodine-labeling method is a Iodine-radiolabeling method.

Under the present invention, the Iodine-labeling method is a direct or an indirect labeling method for obtaining compounds of formula (I), (II) or mixtures thereof.

The Iodine-labeling method comprises the steps
Reacting a compound of general formula (III) with an Iodine containing moiety,
Optionally deprotecting compound of formula (II) and
Optionally converting obtained compound into a suitable salt of inorganic or organic acids thereof, hydrates, complexes and solvates thereof.

The iodine-labeling method comprises the steps
Reacting compound of general Formula (III) with Iodine containing moiety wherein the Iodine is ¹²³I, ¹²⁴I, ¹²⁵I or ¹³¹I,
Optionally removing protecting group(s) of compound of formula (II) and
Optionally converting obtained compound into an acceptable salts of inorganic or organic acids thereof, hydrates, complexes, esters, amides, and solvates thereof.

Preferably, the iodine-labeling method comprises the steps
Reacting compound of general Formula (III) with Iodine containing moiety wherein the Iodine is ¹²³I, ¹²⁴I, ¹²⁵I or ¹³¹I,
Removing protecting group(s) of compound of formula (II) and
Optionally converting obtained compound into an acceptable salts of inorganic or organic acids thereof, hydrates, complexes, esters, amides, and solvates thereof.

The reagents, solvents and conditions which are used for this iodination are common and well-known to the skilled person in the field.

Preferably, the solvents used in the present method is water, aqueous buffer, DMF, DMSO, acetonitrile, DMA, or mixtures thereof, preferably the solvent is water, aqueous buffer or acetonitrile.

Preferably the iodine-labeling method comprises the steps
Reacting compound of general Formula (III) with Iodine containing moiety wherein the Iodine is ¹²³I, or ¹²⁵I, and
Removing protecting group(s) of compound of formula (II) and
Optionally converting obtained compound into an acceptable salts of inorganic or organic acids thereof, hydrates, complexes, esters, amides, and solvates thereof.

Preferably the iodine-labeling method comprises the steps
Reacting compound of general Formula (III) with Iodine containing moiety wherein the Iodine is ¹²⁴I, and
Removing protecting group(s) of compound of formula (II) and
Optionally converting obtained compound into an acceptable salts of inorganic or organic acids thereof, hydrates, complexes, esters, amides, and solvates thereof.

Preferably the iodine-labeling method comprises the steps
Reacting compound of general Formula (III) with Iodine containing moiety wherein the Iodine is ¹³¹I and
Removing protecting group(s) of compound of formula (II) and
Optionally converting obtained compound into an acceptable salts of inorganic or organic acids thereof, hydrates, complexes, esters, amides, and solvates thereof.

Preferably the iodine-labeling method comprises the steps
Reacting compound of general Formula (III) with Iodine containing moiety wherein the Iodine is ¹²⁷I and
Removing protecting group(s) of compound of formula (II) and
Optionally converting obtained compound into an acceptable salts of inorganic or organic acids thereof, hydrates, complexes, esters, amides, and solvates thereof.

Compounds of formula (I), (II) or (III) are as disclosed above.

Embodiments and preferred features can be combined together and are within the scope of the invention. The preferred features disclosed for compound of general formula (I) (II) and (III) are incorporated herein.

In a sixth aspect, the invention is directed to compounds of general formula (I) or (II) for the manufacture of an imaging tracer for imaging proliferative diseases.

In other word, the invention is directed to the use of invention compounds of general formula (I) and (II) for the manufacture of an imaging tracer for imaging proliferative diseases.

The compounds of general formula (I) and (II) are herein defined as above and encompass all embodiments and preferred features. Preferably, the invention compounds are compounds of general formula (I) or (II) wherein the Iodine is ¹²³I, ¹²⁴I, or ¹²⁵I.

The imaging tracer is suitable for Single Photon Emission Computed Tomography (SPECT) , and Positron Emission Tomography (PET).

The imaging tracer is suitable for Single Photon Emission Computed Tomography (SPECT) when the Iodine is ¹²³I, or ¹²⁵I.

The imaging tracer is suitable for Positron Emission Tomography (PET) when the Iodine is ¹²⁴I.

The invention is also directed to a method for imaging or diagnosis proliferative diseases comprising the steps:
Administering to a mammal an effective amount of a compound comprising compounds of general formula (I) or (II) or mixture there of,
Obtaining images of the mammal and
Assessing the images.

Proliferative diseases are cancer characterised by the presence of tumor and/or metastases. Preferably, tumour are selected from the group of malignomas of the gastrointestinal or colorectal tract, liver carcinoma, pancreas carcinoma, kidney carcinoma, bladder carcinoma, thyroid carcinoma, prostrate carcinoma, endometrial carcinoma, ovary carcinoma, testes carcinoma, melanoma, small-cell and non-small-cell bronchial carcinoma, dysplastic oral mucosa carcinoma, invasive oral cancer; breast cancer, including hormone-dependent and hormone-independent breast cancer, squamous cell carcinoma, neurological cancer disorders including neuroblastoma, glioma, astrocytoma, osteosarcoma, meningioma, soft tissue sarcoma; haemangioma and endocrine tumours, including pituitary adenoma, chromocytoma, paraganglioma, haematological tumour disorders including lymphoma and leukaemias; Preferably, the tumor is prostrate carcinoma.

Preferably, metastases are metastases of one of the tumours mentioned above.

Preferably, the invention compounds and use is for manufacturing a SPECT imaging tracer for imaging tumor in a mammal wherein the tumor is preferably a prostate carcinoma/prostate tumor.

In a seventh aspect, the invention is directed to the use of compounds of general formula (I), (II) or (III) for conducting biological assays and chromatographic identification. More preferably, the use relates to compounds of general formula (I) or (II) wherein the iodine isotope is ¹²³I, ¹²⁴I, ¹²⁵I, or ¹³¹I, more preferably ¹²⁵I.

Compounds of general formula (I), (II) or (III) wherein the iodine isotope (I) is ¹²⁷I are useful as reference and/or measurement agent.

The compounds of general formula (I), (II) and (III) are herein defined as above and encompass all embodiments and preferred features.

In an eighth aspect, the present invention provides a kit comprising a sealed vial containing a predetermined quantity of a compound having general chemical Formula (I), (II) or (III) and suitable salts of inorganic or organic acids thereof, hydrates, complexes, esters, amides, and solvates thereof. Optionally the kit comprises a pharmaceutically acceptable carrier, diluent, excipient or adjuvant.

In a ninth aspect, the present invention is directed to compounds of general formula (I) or (II) for the manufacture of a medicament for radiotherapy of proliferative diseases wherein the iodine isotope is ¹³¹I.

### Definitions

The terms used in the present invention are defined below but are not limiting the invention scope.

If chiral centers or other forms of isomeric centers are not otherwise defined in a compound according to the present invention, all forms of such stereoisomers, including enantiomers and diastereoisomers, are intended to be covered herein. Compounds containing chiral centers may be used as racemic mixture or as an enantiomerically enriched mixture or as a diastereomeric mixture or as a diastereomerically enriched mixture, or these isomeric mixtures may be separated using well-known techniques, and an individual stereoisomer maybe used alone. In cases in which compounds have carbon-carbon double bonds, both the (Z)-isomers and (E)-isomers as well as mixtures thereof are within the scope of this invention. In cases wherein compounds may exist in tautomeric forms as it is the case e.g. in tetrazole derivatives, each tautomeric form is contemplated as being included within this invention whether existing in equilibrium or predominantly in one form.

Suitable salts of the compounds according to the invention include salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disul-phonic acid, acetic acid, trifluoroacetic acid, propionic acid, lactic acid, tartaric acid, malic acid, citric acid, fumaric acid, maleic acid and benzoic acid.

Suitable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyl [symbol] diiso [symbol] propyl [symbol] amine, monoethanolamine, diethanolamine, triethanolamine, dicyclo [symbol] hexylamine, dimethylaminoethanol, procaine, diben-zylamine, N-methyl [symbol] morpholine, argin [symbol] ine, lysine, ethylenediamine and N-methylpiperidine.

The term “C₁-C₅ alkyl”, used herein on its own or as part of another group, refers to saturated carbon chains which may be straight-chain or branched, in particular to methyl, ethyl, n-propyl, isopropyl, n-butyl, isobutyl, tert-butyl, methylpropyl, n-pentyl, 2,2-dimethylpropyl, 2-methylbutylor 3-methylbutyl. Preferably, alkyl is methyl, ethyl, propyl, butyl or n-pentyl.

The term “aryl” as employed herein by itself or as part of another group refers to mono or bicyclic C₆-C₁₀ aromatic rings, in particular phenyl or naphthyl groups e.g. 1-naphthyl and 2-naphthyl, which themselves can be substituted with one, two or three substituents independently and individually selected from but not limited to the group comprising OH, NH₂, protected amino, (C₁-C₃)alkyl (C₁-C₃)alkoxy.

The term “heteroaryl” as employed herein by itself or as part of another group refers to heteroaromatic groups containing from 5 to 6 ring atoms, wherein 1 or 2 atoms of the ring portion are independently selected from N, O or S, e.g. thienyl, furanyl, pyrrolyl, oxazolyl, thiazolyl, imidazolyl, pyrazolyl, isoxazolyl, isothiazolyl, pyridyl, pyridazinyl, pyrimidinyl, pyrazinyl etc.; which themselves can be substituted with one methyl group.

Halogen as used herein refers to fluoro, chloro, bromo or iodo.

B means Boron.

The term “amine-protecting group” as employed herein by itself or as part of another group is known or obvious to someone skilled in the art, which is chosen from but not limited to a class of protecting groups namely carbamates, amides, imides, N-alkyl amines, N-aryl amines, imines, enamines, boranes, N-P protecting groups, N-sulfenyl, N-sulfonyl and N-silyl, and which is chosen from but not limited to those described in the textbook Greene and Wuts, Protecting groups in Organic Synthesis, third edition, page 494-653, included herewith by reference.

Amino protecting groups are selected e.g. from the group comprising Carbobenzyloxy (Cbz), tert-Butyloxycarbonyl (BOC) or 9-Fluorenylmethyloxycarbonyl (FMOC).

O-protecting groups are selected e.g. from the group comprising

Methyl, Ethyl, Propyl, Butyl, t-Butyl or Benzyl.

Unless otherwise specified, when referring to the compounds of formula the present invention per se as well as to any pharmaceutical composition thereof the present invention includes all of the hydrates, salts, and complexes.

### General Synthesis of Radioiodo Compounds: aryl-1 And (hetero)aryl-I

SPECT detectable radio iodo isotopes can be introduced into compounds by the following published methods.

The radioiodination reaction can be carried out, for example in a typical reaction vessel (e.g. Wheaton vial, Eppendorf vial, Iodogen tube etc.) which is known to someone skilled in the art or in a microreactor. Typically the reactions are carried out at room temperature in aqueous solutions. These aqueous solutions can contain but are not limited to acids and buffers. If necessary for a quicker conversion the reactions (e.g. radioiodo-dehalogenations or radioiodo-detriazenation) can be carried out in a sealed vial under elevated temperatures. Therefore the vial can be heated by typical methods, e.g. oil bath, heating block or microwave. In the case of electrophilic radioiodination substitution reactions the generation of an electrophilic iodine species is carried out in-situ by the addition of a suitable oxidizing agent. These oxidizing agents can be taken from but are not limited to the group of N-chloramides, hydrogen peroxide, Iodogen, N-halosuccinimides and peracids. These in situ oxidations can e.g. be used for direct iodo-deprotonations, iodo-demetallations or indirect iodinations with heterobifunctional reagents like 4-hydroxyphenyl succinimidyl esters (Bolton and Hunter reagent; Biochem. J. 1973, 133, 529). Organic solvents can be involved in such a reaction as co-solvent. The radioiodination reactions are conducted for one to 60 minutes. This and other conditions for such radioiodinations are known to experts (Eisenhut M., Mier W., Radioiodination Chemistry and Radioiodinated Compounds (2003) in: Vertes A., Nagy S., Klenscar Z., (eds.) Rösch F. (volume ed.), Handbook of Nuclear Chemistry, 4, pp. 257-278 and Coenen H. H., Mertens J., Mazière B., Radioiodination Reactions for Pharmaceuticals, pp. 29-72).

Precursors for aryl-radioiodo compounds of general formula I and II are e.g. the iodine free compounds of formula (I) or compounds of formula (III) with or without electron-donating groups at the aryl ring. The aryl compounds without electron-donating groups can e.g. be radioiodinated via radioiodo-dethallation (e.g. J. Nucl. Med. 2000, 38, 1864). The corresponding electron-donating group substituted aryl compounds can e.g. be directly radioiodinated with the aid of an oxidizing agent like chloramine-T (e.g. J. Med. Chem. 1988, 31, 1039), iodogen (e.g. J. Biol. Chem. 1990, 265, 14008), peracetic acid (e.g. J. Nucl. Med. 1991, 32, 339), lactoperoxidase (e.g. Meth. Enzymol. 1980, 70, 214) and others.

Other precursors of general formula III for aryl-radioiodo compounds of general formula I and II are e.g. arylstannyl compounds (e.g. Nucl. Med. Biol. 1993, 20, 597), arylboronic acids (e.g. U.S. 2008/312459) or aryl-triazenes (e.g. J. Med. Chem. 1984, 27, 156). Starting materials for these precursors are commercially available or can be synthesized by methods known in the art (R. C. Larock, Comprehensive Organic Transformations, VCH Publishers 1989).

Precursors for the aryl-radioiodo compounds of general formula I and II can also be e.g. arylhalogenated compounds like aryliodides (e.g. J. Org. Chem. 1982, 47, 1484) or arylbromides (e.g. J. Labeled Comp. Radiopharm. 1986, 23, 1239).

The radioiodinated compounds of general formula I and II can also be build up via an indirect labeling method using a prosthetic group like the Bolton-Hunter-reagent (Biochem. J. 1973, 133, 529) and others.

Precursors for the heteroaryl-radioiodo compounds of general formula I and II can be the corresponding iodine free compounds of formula (I) or compounds of formula (III), the halogenated compounds, the heteroaryl stannyl compounds or the heteroaryl boronic acids. These precursors can be converted to the corresponding radioiodinated products as cited above for the aryl-radioiodo compounds.

Precursors for the vinyl-radioiodo compounds of general formula I can be e.g. vinyl-trialkylsilyl compounds (e.g. J. Med. Chem. 1997, 40, 2184), vinyltrialkylstannyl compounds (e.g. J. Labeled Comp. Radiopharm. 1998, 41, 801), vinylboronic acids (e.g. J. Med. Chem. 1984, 27, 1287), alkinyl compounds that can be converted to suitable vinyl compounds via hydroborination with e.g. catecholborane (e.g. J. Med. Chem. 1984, 27, 57), hydrostannylation with e.g. HSnBu₃ (e.g. J. Med. Chem. 1995, 38, 3908) and other conversions.

### Experimental Section

### Abbreviations

| | |
|---|---|
| br | broad signal (in NMR) |
| d | doublet |
| dd | doublet of doublet |
| DMA | N,N-dimethylacetamide |
| DMF | N,N-dimethylformamide |
| DMSO | dimethylsulphoxide |
| dt | doublet of triplet |
| EE | Ethyl acetate |
| ESI | Electrospray ionisation |
| Hex | Hexane |
| MS | Mass spectrometry |
| m | multiplet |
| NMR | Nuclear magnetic resonance |
| | spectroscopy: chemical shifts (δ) are |
| | given in ppm. |
| r.t. | room temperature |
| s | Singlet |
| t | Triplet |
| THF | Tetrahydrofurane |
| TFA | Trifluoro acetic acid |

### EXAMPLES

### Example 1

### (2S,4S)-2-Amino-4-(4-hydroxy-3-[I-125]-iodobenzyl)-pentanedioic acid

a) Di-tert-butyl (2S,4S)-4-(4-benzyloxy)benzyl-2-tert-butoxycarbonylamino-pentane-dioate

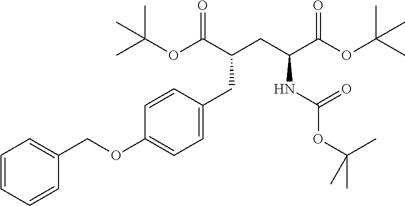 [SMILES: CC(C)(C)OC(=O)N[C@@H](C[C@H](Cc1ccc(OCc2ccccc2)cc1)C(=O)OC(C)(C)C)C(=O)OC(C)(C)C]

2.16 g (6 mmol) of Di-tert-butyl Boc-glutamate (Journal of Peptide Research (2001), 58, 338) were dissolved in 18 mL of tetrahydrofuran (THF) and cooled to −70° C. 13 mL (13 mmol) of a 1M solution of lithium bis(trimethylsilyl)amide in tetrahydrofuran were added dropwise at this temperature and the mixture was stirred at −70° C. for another 2 hours. 5.0 g (18 mmol) of 4-benzyloxybenzyl bromide in 15 mL of THF were then added dropwise, and after 2 h at this temperature, the cooling bath was removed and 150 mL of 2N aqueous hydrochloric acid and 500 mL of dichloromethane were added. The organic phase was separated off, washed with water until neutral, dried over sodium sulphate and filtered, and the filtrate was concentrated. The crude product obtained in this manner was chromatographed in silica gel using a hexane/ethyl acetate gradient, and the appropriate fractions were combined and concentrated.

Yield: 0.48 g (12.5%)

MS (ESIpos): m/z=556 [M+H]⁺

1H NMR (300 MHz, CHLOROFORM-d) d ppm 1.32 (s, 9H), 1.44-1.45 (m, 18H), 1.86-1.91 (t, 2H), 2.60-2.64 (m, 1H), 2.79-2.82 (m, 2H), 4.15-4.22 (m, 1H), 4.87-4.90 (m, 1H), 5.05 (s, 2H), 6.87-6.89 (m, 2H), 7.08-7.10 (m, 2H), 7.36-7.44 (m, 5H)

b) Di-tert-butyl (2S,4S)-4-(4-hydroxy)benzyl-2-tert-butoxycarbonylamino-pentanedioate

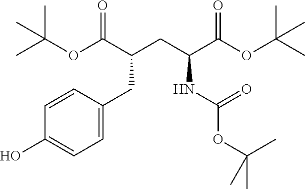 [SMILES: CC(C)(C)OC(=O)N[C@@H](C[C@H](Cc1ccc(O)cc1)C(=O)OC(C)(C)C)C(=O)OC(C)(C)C]

340 mg (0.61 mmol) of Di-tert-butyl (2S,4S)-4-(4-benzyloxy)benzyl-2-tert-butoxy-carbonylamino-pentanedioate (1a) were dissolved in 20 mL of methanol. 170 mg of palladium on charcoal (10%) were added and the suspension was hydrogenated overnight at room temperature. After filtration from the catalyst the filtrate was concentrated and the crude product obtained in this manner was chromatographed in silica gel using a hexane/ethyl acetate gradient, and the appropriate fractions were combined and concentrated.

Yield: 186 mg (64.0%)

MS (ESIpos): m/z=466 [M+H]⁺

1H NMR (500 MHz, CHLOROFORM-d) d ppm 1.34 (s, 9H), 1.45-1.46 (m, 18H), 1.87-1.90 (t, 2H), 2.60-2.63 (m, 1H), 2.78-2.81 (m, 2H), 4.18-4.20 (m, 1H), 4.86-4.90 (m, 2H), 6.72-6.74 (m, 2H), 7.03-7.05 (m, 2H)

c) (2S,4S)-4-(4-hydroxy)benzyl-2-amino-pentanedioic acid

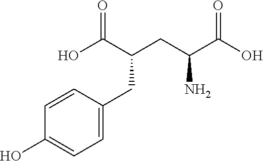 [SMILES: N[C@@H](C[C@H](Cc1ccc(O)cc1)C(=O)O)C(=O)O]

90 mg (0.193 mmol) of di-tert-butyl (2S,4S)-4-(4-hydroxy)benzyl-2-tert-butoxycarbonylamino-pentanedioate (1b) were dissolved in 2 mL of dichloromethane and 2 mL of trifluoroacetic acid and stirred for 3 days at room temperature. The reaction mixture was then evaporated to dryness and the resulting crude product was then chromatographed with water/methanol on C18-silica gel and the resulting fractions were combined and reduced in volume by evaporation.

Yield: 20 mg (40.9%)

MS (ESIpos): m/z=254 [M+H]⁺

1H NMR (400 MHz, DMSO-d6) d ppm 1.64-1.68 (t, 2H), 2.38-2.43 (m, 1 H), 2.74-2.87 (m, 2H), 3.44-3.49 (m, 1H), 6.64-6.66 (m, 2H), 6.94-6.96 (m, 2H), 9.17 (br, 1H)

d) (2S,4S)-2-Amino-4-(4-hydroxy-3-[I-125]odobenzyl)-pentanedioic acid

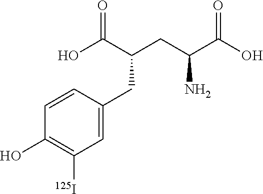 [SMILES: N[C@@H](C[C@H](Cc1ccc(O)c([125I])c1)C(=O)O)C(=O)O]

0.5 mg of (2S,4S)-4-(4-hydroxy)benzyl-2-amino-pentanedioic acid was dissolved in 1 mL of PBS buffer and transferred to a vial coated with 500 μg of Iodogen™. To this mixture 10 μL of a solution of 0.1 N [¹²⁵I]NaI (81 MBq) in 0.1 N NaOH was added and stirred for 15 min at 25° C. The reaction mixture was poured into another vial, diluted with 4 mL water/acetonitrile (2/1 v/v) and subsequently transferred to the HPLC unit using a remote-control-operated HPLC injection system and subjected to a semi-preparative HPLC purification using a Agilent Zorbax Bonus-RP C18, 5 μm; 250_{—}9.4 mm column. Eluent was acetonitrile/water with 0.1% trifluoroacetic acid at a flow of 4 ml/min. For the purification a linear gradient from 20 to 80% acetonitrile within 20 min was used. The HPLC fraction containing the product peak was neutralized with 0.5 M NaOH and passed through a sterile filter to get in 5.5 mL 67 MBq of the final tracer in a radiochemical yield of 82% and a radiochemical purity of 99% after a synthesis time of 83 min.

### Example 2

### (2S,4S)-2-Amino-4-(4-hydroxy-3-iodobenzyl)-pentanedioic acid

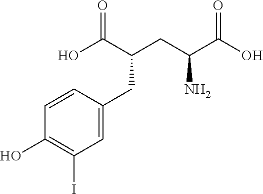 [SMILES: N[C@@H](C[C@H](Cc1ccc(O)c(I)c1)C(=O)O)C(=O)O]

10 mg (0.039 mmol) of (2S,4S)-4-(4-hydroxy)benzyl-2-amino-pentanedioic acid in 0.7 mL aqueous ammonia were cooled in an ice-bath. 10 mg (0.039 mmol) of iodine in 0.1 mL of ethanol were then added dropwise to the solution. The organic solvent was then evaporated and the resulting aqueous solution was acidified with concentrated hydrochloric acid to pH 4.5. The resulting precipitate was separated off and the filtrate was evaporated to dryness and the resulting crude product was then chromatographed with water/methanol on C18-silica gel and the resulting fractions were combined and reduced in volume by evaporation.

Yield: 9 mg (57.1%)

MS (ESIpos): m/z=380 [M+H]⁺

1H NMR (300 MHz, D₂O) d ppm 1.68-4.06 (m, 6H), 6.81-6.86 (m, 1 H), 7.03-7.09 (m, 1 H), 7.58-7.60 (m, 1H)

### Example 3

### (2S,4S)-2-Amino-4-(3-[4-[I-125]-iodophenoxy]propyl)-pentanedioic acid

a) Di-tert-butyl (2S,4S)-4-Allyl-2-tert-butoxycarbonylamino-pentanedioate

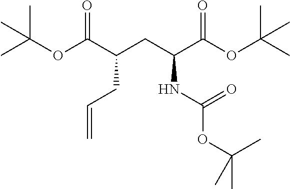 [SMILES: C=CC[C@@H](C[C@H](NC(=O)OC(C)(C)C)C(=O)OC(C)(C)C)C(=O)OC(C)(C)C]

26.96 g (75 mmol) of di-tert-butyl Boc-glutamate (Journal of Peptide Research (2001), 58, 338) were dissolved in 220 mL of tetrahydrofuran (THF) and cooled to −70° C. 165 mL (165 mmol) of a 1M solution of lithium bis(trimethylsilyl)amide in THF were added dropwise over a period of two hours at this temperature and the mixture was stirred at -70° C. for another 2 hours. 27.22 g (225 mmol) of allyl bromide were then added dropwise, and after 2 h at this temperature, the cooling bath was removed and 375 mL of 2N aqueous hydrochloric acid and 1.25 L of ethyl acetate were added. The organic phase was separated off, washed with water until neutral, dried over sodium sulphate and filtered, and the filtrate was concentrated. The crude product obtained in this manner was chromatographed in silica gel using a hexane/ethyl acetate gradient, and the appropriate fractions were combined and concentrated.

Yield: 15.9 g (53.1%)

MS (ESIpos): m/z=400 [M+H]⁺

1H NMR (300 MHz, CHLOROFORM-d) d ppm 1.32-1.58 (m, 27H) 1.81-1.92 (m, 2H) 2.25-2.39 (m, 2H) 2.40-2.48 (m, 1 H), 4.10-4.18 (m, 1 H) 4.85-4.92 (d, 1H) 5.02-5.11 (m, 2H) 5.68-5.77 (m, 1H)

b) Di-tert-butyl (2S,4S)-2-tert-butoxycarbonylamino-4-(3-hydroxypropyl)-pentanedioate

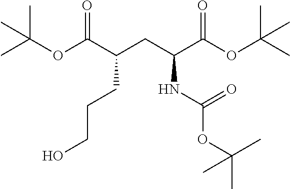 [SMILES: CC(C)(C)OC(=O)N[C@@H](C[C@H](CCCO)C(=O)OC(C)(C)C)C(=O)OC(C)(C)C]

15.58 g (39 mmol) of the compound described in Example 3a were dissolved in 200 mL of tetrahydrofuran and cooled in an ice-bath. Over a period of about 20 minutes, 54.6 mL (54.6 mmol) of 1 M diboran/tetrahydrofuran complex in tetrahydrofuran were added dropwise with ice-cooling and under nitrogen, and the mixture was stirred on ice for 2 h and at room temperature overnight. It was cooled again to 0° C. and 58.5 mL of 1 N aqueous sodium hydroxide solution and 58.5 mL of 30% aqueous hydrogen peroxide solution were then added dropwise. After 30 minutes, the mixture was diluted with water, the tetrahydrofuran was distilled off and the remaining aqueous solution was extracted with ethyl acetate. The organic phase was separated off, washed with water until neutral, dried over sodium sulphate and filtered, and the filtrate was concentrated. The crude product obtained in this manner was chromatographed on silica gel using a hexane/ethyl acetate gradient, and the appropriate fractions were combined and concentrated.

Yield: 8.5 g (52.2%

MS (ESIpos): m/z=418 [M+H]⁺

1H NMR (300 MHz, CHLOROFORM-d) d ppm 1.32-1.58 (m, 27H) 1.60-1.70 (m, 2H) 1.73-1.94 (m, 4H) 2.05-2.12 (m, 1H), 2.33-2.40 (m, 1H) 3.58-3.68 (m, 2H) 4.15-4.22 (m, 1H) 4.95-5.03 (d, 1H)

c) Di-tert-butyl (2S,4S)-2-tert-butoxycarbonylamino-4-(3-[4-iodophenoxy]propyl)-pentanedioate

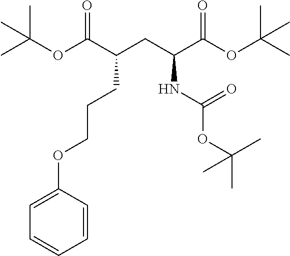 [SMILES: CC(C)(C)OC(=O)N[C@@H](C[C@H](CCCOc1ccccc1)C(=O)OC(C)(C)C)C(=O)OC(C)(C)C]

4.18 g (10 mmol) of di-tert-butyl (2S,4S)-2-tert-butoxycarbonylamino-4-(3-hydroxypropyl)pentanedioate (3b) were dissolved in 100 mL of THF and cooled in an ice-bath. After addition of 0.94 g (10 mmol) of phenol and 3.67 g (14 mmol) of triphenyl phosphine, 2.92 g (2.60 mL, 18.8 mmol) of diethyl azodicarboxylate were added. The mixture was stirred on ice for 2 h and overnight at room temperature, then concentrated. The crude product obtained in this manner was chromatographed on silica gel using a hexane/ethyl acetate gradient and the appropriate fractions were combined and concentrated.

Yield: 2.1 g (42.5%)

MS (ESIpos): m/z=494 [M+H]⁺

1H NMR (300 MHz, CHLOROFORM-d) d ppm 1.44 (s, 9H), 1.46-1.48 (m, 18H) 1.60-2.01 (m, 6H) 2.38-2.42 (m, 1H) 3.94-3.96 (m, 3H), 4.02-4.24 (m, 1H) 4.87-4.90 (m, 1H) 5.30-5.31 (m, 1H) 6.87-6.98 (m, 3H), 7.25-7.30 (m, 2H)

d) (2S,4S)-2-Amino-4-(3-phenoxy]propylypentanedioic acid

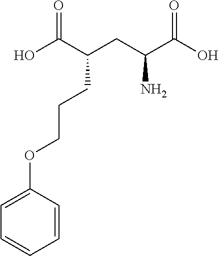 [SMILES: N[C@@H](C[C@H](CCCOc1ccccc1)C(=O)O)C(=O)O]

987 mg (2 mmol) of di-tert-butyl (2S,4S)-2-tert-butoxycarbonylamino-4-(3-[4-iodophenoxy]propyl)-pentanedioate (3c) were dissolved in 20 mL of methoxybenzene and 10 mL of trifluoroacetic acid and stirred overnight at room temperature. The reaction mixture was then evaporated to dryness and the resulting crude product was then chromatographed with water/methanol on C18-silica gel and the resulting fractions were combined and reduced in volume by evaporation.

Yield: 0.3 g (53%)

MS (ESIpos): m/z=282 [M+H]⁺

1H NMR (300 MHz, DMSO-d6) d ppm 1.39-1.76 (m, 6H) 2.67-2.78 (m, 1H) 3.33-3.50 (m, 3H) 3.82-4.02 (m, 2H) 6.89-6.92 (m, 3H), 7.24-7.29 (m, 2H)

e) (2S,4S)-2-Amino-4-(3-[4-[I-125]-iodophenoxy]propylypentanedioic acid

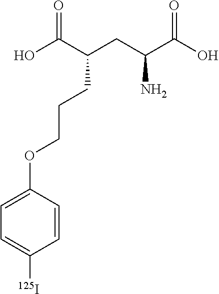 [SMILES: N[C@@H](C[C@H](CCCOc1ccc([125I])cc1)C(=O)O)C(=O)O]

20 μL of a 10 mM trifluoroacetic acid (TFA) solution of (2S,4S)-2-amino-4-(3-phenoxy]propyl)-pentanedioic acid was mixed with 10 μL of 10 mM thallium-(III)-tris-trifluoroacetate dissolved in TFA. After 10 min stirring at 25° C. the solution 2 μL of a solution of 0.1 N [¹²⁵I]NaI (35.9 MBq) in 0.1 N NaOH was added to the reaction mixture and stirred for additional 5 min at 25° C. The reaction mixture was poured into another vial, diluted with 4 mL water and subsequently transferred to the HPLC unit using a remote-control-operated HPLC injection system and subjected to a semi-preparative HPLC purification using a Agilent Zorbax Bonus-RP C18, 5 μm; 250_{—}9.4 mm column. Eluent was acetonitrile/water with 0.1% trifluoroacetic acid at a flow of 4 ml/min. For the purification a linear gradient from 20 to 80% acetonitrile within 20 min was used. The HPLC fraction containing the product peak was neutralized with 0.5 M NaOH and passed through a sterile filter to get in 2.4 mL 18.2 MBq of the final tracer in a radiochemical yield of 51% and a radiochemical purity of 98% after a synthesis time of 102 min.

### Example 4

### (2S,4S)-2-Amino-4-(3-[4-iodophenoxy]propyl)-pentanedioic acid

a) Di-tert-butyl (2S,4S)-2-tert-butoxycarbonylamino-4-(3-[4-iodophenoxy]propyl)-pentanedioate

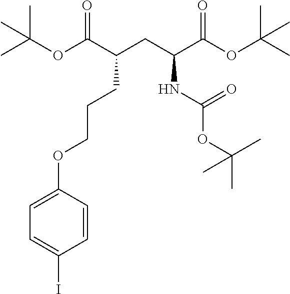 [SMILES: CC(C)(C)OC(=O)N[C@@H](C[C@H](CCCOc1ccc(I)cc1)C(=O)OC(C)(C)C)C(=O)OC(C)(C)C]

2.92 g (7 mmol) of di-tert-butyl (2S,4S)-2-tert-butoxycarbonylamino-4-(3-hydroxypropyl)pentanedioate (3b) were dissolved in 50 mL of THF and cooled in an ice-bath. After addition of 1.10 g (5 mmol) of 4-iodophenol and 1.84 g (7 mmol) of triphenyl phosphine, 1.46 g (1.3 mL, 8.4 mmol) of diethyl azodicarboxylate were added. The mixture was stirred on ice for 2 h and overnight at room temperature, then concentrated. The crude product obtained in this manner was chromatographed on silica gel using a hexane/ethyl acetate gradient and the appropriate fractions were combined and concentrated.

Yield: 1.0 g (32.3%)

MS (ESIpos): m/z=620 [M+H]⁺

1H NMR (400 MHz, CHLOROFORM-d) d ppm 1.43-1.46 (m, 27H) 1.73-1.90 (m, 6H) 2.38-2.41 (m, 1H) 3.90-3.93 (m, 1H) 4.12-4.17 (m, 2H) 4.89 (d, 1H) 6.63-6.69 (m, 2H) 7.50-7.56 (m, 2H)

b) (2S,4S)-2-Amino-4-(3-[4-iodophenoxy]propyl)-pentanedioic acid

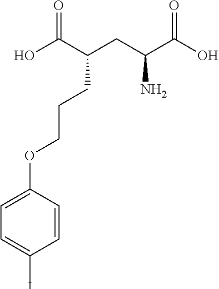 [SMILES: N[C@@H](C[C@H](CCCOc1ccc(I)cc1)C(=O)O)C(=O)O]

929 mg (11.5 mmol) of di-tert-butyl (2S,4S)-2-tert-butoxycarbonylamino-4-(3-[4-iodophenoxy]propyl)-pentanedioate (4a) were dissolved in 20 mL of trifluoroacetic acid and stirred overnight at room temperature. The reaction mixture was then evaporated to dryness and the resulting crude product was then chromatographed with water/methanol on C18-silica gel and the resulting fractions were combined and reduced in volume by evaporation.

Yield: 0.32 g (52.4%)

MS (ESIpos): m/z=408 [M+H]⁺

1H NMR (300 MHz, DMSO-d6) d ppm 1.33-1.73 (m, 6H) 2.55-2.69 (m, 1H) 3.37-3.43 (m, 3H) 3.85-3.89 (m, 2H) 6.71-6.75 (m, 2H), 7.50-7.55 (m, 2H)

### Example 5

Biological characterisation. The ability of compounds from the present invention to bind to tumor cells was investigated in several cell-experiments.

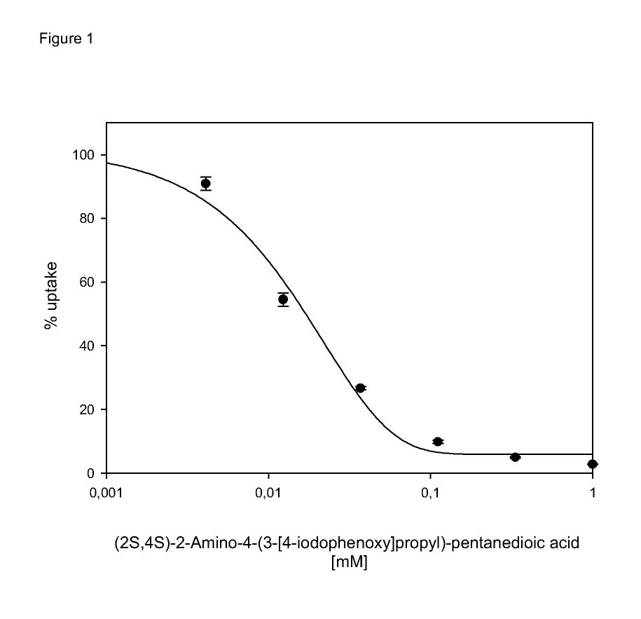

The specificity of binding to NCl-H460 (human NSCLC) tumor cells was examined using 3H-Glutamic acid as tracer and (2S,4S)-2-Amino-4-(3-[4-iodophenoxy]propyl)-pentanedioic acid in concentrations ranging from 4 μM to 1 mM. Surprisingly, (2S,4S)-2-Amino-4-(3-[4-iodophenoxy]propyl)-pentanedioic acid was able to reduce the uptake of glutamic acid in NCl-H460 cells in a concentration dependent manner, indicating that the same transport systems may be exploited by the iodinated compound (FIG. 1).

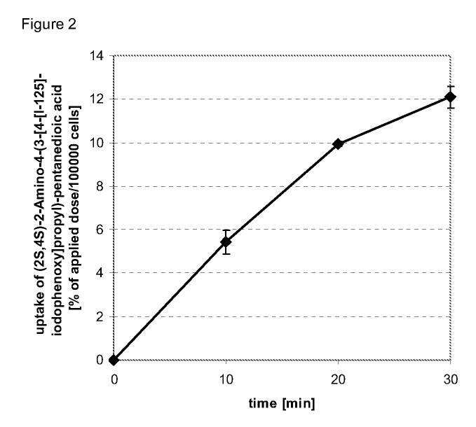

In a next experiment, NCl-H460 cells were incubated with [I125]-labeled (2S,4S)-2-Amino-4-(3-[4-[I-125]-iodophenoxy]propylypentanedioic acid for up to 30 min and the cell-bound fraction was determined. Approximately 12% of applied activity was bound to the cells after 30 min incubation (FIG. 2).

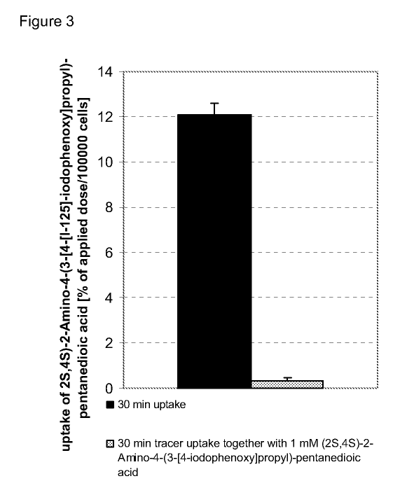

Furthermore, the specificity of binding was examined using (2S,4S)-2-Amino-4-(3-[4-[I-125]-iodophenoxy]propylypentanedioic acid as tracer and (2S,4S)-2-Amino-4-(3-[4-iodophenoxy]propyl)-pentanedioic acid in excess (1 mM) to compete for binding sites. Interestingly, a large decrease in binding was observed (FIG. 3).

### Example 6

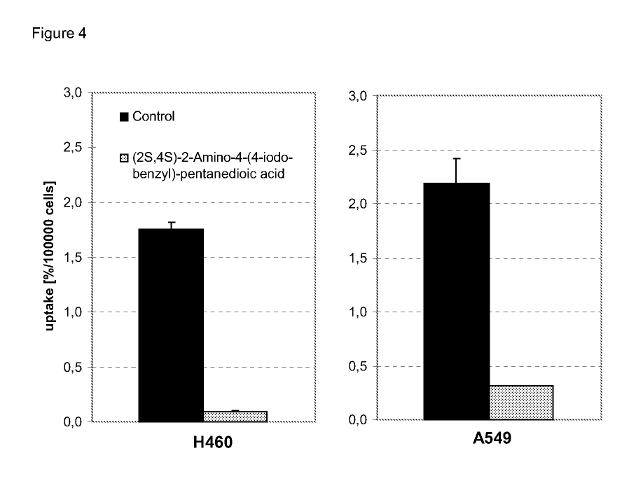

The specificity of binding was examined in a cell competition experiment using 3H-glutamic acid as tracer and (2S,4S)-2-Amino-4-(4-iodo-benzyl)-pentanedioic acid in excess (1 mM) to compete for transporter. Interestingly, the tested compound was able to reduce the uptake of glutamic acid in A549 (human NSCLC cell line) as well as in NCl-H460 (human NSCLC) cells, indicating that the same transport systems may be exploited by the test-compound (FIG. 4).

### Example 7

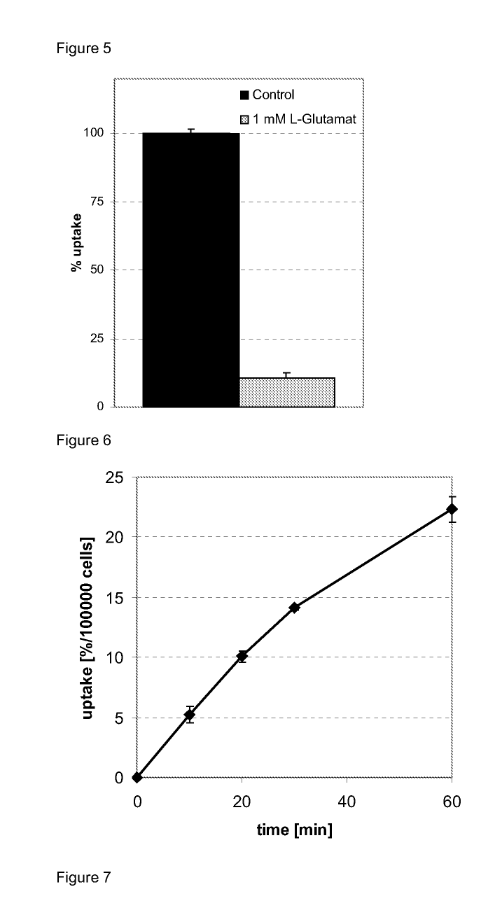

To determine the specificity of (2S,4S)-2-Amino-4-(4-hydroxy-3-[1-125]-iodobenzyl)-pentanedioic acid, the compound was used as tracer in a cell competition experiment in H460 tumor cells against an excess of L-Glutamic acid (1 mM). Interestingly, it was discovered, that the uptake was blockable by excess of glutamic acid, indicating the potential use of the same uptake system (FIG. 5).

FIG. 1: Concentration dependent blocking of 3H-Glutamic acid uptake in H460 cells using different concentrations of (2S,4S)-2-Amino-4-(3-[4-iodophenoxy]propyl)-pentanedioic acid.

FIG. 2: E xamination of biological activity of (2S,4S)-2-Amino-4-(3-[4-[I-125]-iodophenoxy]propylypentanedioic acid in a tumor cell uptake/binding experiment. (NCl-H460 cells, up to 30 min incubation with I125-labeled derivative).

FIG. 3: Examination of biological activity of (2S,4S)-2-Amino-4-(3-[4-[I-125]-iodophenoxy]propylypentanedioic acid in a cell competition experiment. (NCl-H460 cells, 30 min incubation with I125-labeled derivative in PBS-buffer, concentration of “cold” derivative 1 mM).

FIG. 4: Examination of biological activity of (2S,4S)-2-Amino-4-(4-iodo-benzyl)-pentanedioic acid in a cell competition experiment. (NCl-H460 cells, A549 cells, 10 min incubation with 1 μCi 3H-Glutamic acid in PBS-buffer, concentration of test compound 1 mM).

FIG. 5: Determination of biological activity of (2S,4S)-2-Amino-4-(4-hydroxy-3-[I-125]-iodobenzyl)-pentanedioic acid in a cell competition experiment. (NCl-H460 cells, 10 min incubation with [I125]-labeled derivative in PBS-buffer, concentration of L-Glutamate 1 mM).

### Example 8

### (2S,4S)-2-Amino-4-(4-iodo-benzyl)-pentanedioic acid

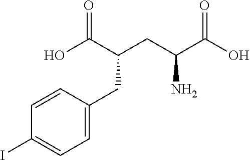 [SMILES: N[C@@H](C[C@H](Cc1ccc(I)cc1)C(=O)O)C(=O)O]

8a) (2S,4S)-2-tert-Butoxycarbonylamino-4-(4-iodo-benzyl)pentanedioic acid di-tert-butyl ester

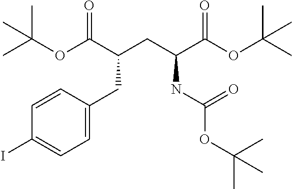 [SMILES: CC(C)(C)OC(=O)N[C@@H](C[C@H](Cc1ccc(I)cc1)C(=O)OC(C)(C)C)C(=O)OC(C)(C)C]

1.44 g (4 mmol) of Di-tert-butyl Boc-glutamate (Journal of Peptide Research (2001), 58, 338) were dissolved in 40 mL of tetrahydrofuran (THF) and cooled to −70° C. 10.4 mL (10.4 mmol) of a 1M solution of lithium bis(trimethylsilyl)amide in tetrahydrofuran were added dropwise at this temperature and the mixture was stirred at −70° C. for another 2 hours. 1.85 g (6.2 mmol) of 4-iodobenzyl bromide in 4 mL of THF were then added dropwise, and after 2 h at this temperature, the cooling bath was removed and 20 mL of 2N aqueous hydrochloric acid and 250 mL of dichloromethane were added. The organic phase was separated off, washed with water until neutral, dried over sodium sulphate and filtered, and the filtrate was concentrated. The crude product obtained in this manner was chromatographed in silica gel using a hexane/ethyl acetate gradient, and the appropriate fractions were combined and concentrated.

Yield: 0.84 g (36.6%)

MS (ESIpos): m/z=576 [M+H]⁺

1H NMR (400 MHz, CHLOROFORM-d) δ ppm 1.31 (s, 9H), 1.44 (m, 18H), 1.79-1.92 (m, 2H), 2.05-2.39 (m, 2H), 2.76-2.86 (m, 2H), 4.17-4.19 (m, 2H), 5.03-5.06 (m, 2H), 6.92-6.95 (m, 2H), 7.56-7.59 (m, 2H)

8b) (2S,4S)-2-Amino-4-(4-iodo-benzyl)-pentanedioic acid

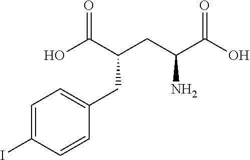 [SMILES: N[C@@H](C[C@H](Cc1ccc(I)cc1)C(=O)O)C(=O)O]

49 mg (0.085 mmol) of di-tert-butyl (2S,4S)-2-tert-Butoxycarbonylamino-4-(4-iodo-benzyl)-pentanedioate (8a) were dissolved in 1 mL of trifluoroacetic acid and stirred for 3 h at room temperature. The reaction mixture was then evaporated to dryness and the resulting crude product was then chromatographed with water/methanol on C18-silica gel and the resulting fractions were combined and reduced in volume by evaporation.

Yield: 28 mg (90.5%)

MS (ESIpos): m/z=364 [M+H]⁺

1H NMR (400 MHz, DMSO-d6) δ ppm 1.73-1.78 (m, 1H), 1.93-1.96 (m, 1H), 2.77-2.89 (m, 3H), 3.82-3.86 (t, 1H), 7.01-7.03 (m, 2H), 7.64-7.66 (m, 2H), 8.23 (br, 3H)

### Example 9

### (2S,4S)-2-tert-Butoxycarbonylamino4-(4-tributylstannanyl-benzyl)-pentanedioic acid di-tert-butyl ester

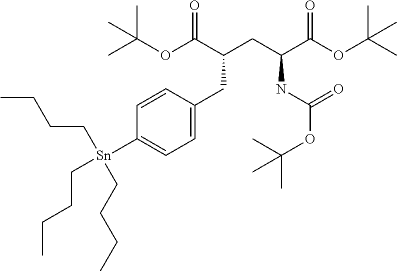 [SMILES: CCC[CH2][Sn]([CH2]CCC)([CH2]CCC)[c]1ccc(C[C@@H](C[C@H](NC(=O)OC(C)(C)C)C(=O)OC(C)(C)C)C(=O)OC(C)(C)C)cc1]

777 mg (1.35 mmol) of (2S,4S)-2-tert-Butoxycarbonylamino-4-(4-iodo-benzyl)-pentanedioic acid di-tert-butyl ester (8a) were dissolved in 30 mL of toluene under nitrogen. 2.34 g (4.03 mmol) of hexabutyldistannane and 17.3 mg (0.015 mmol) of tetrakis(triphenylphosphine) palladium(0) in tetrahydrofuran were added and the mixture was stirred at 60° C. for 3 days. The resulting suspension was filtered and the almost colorless filtrate was concentrated in vacuo and immediately after chromatographed on silica gel using a hexane/ethyl acetate gradient, and the appropriate fractions were combined and concentrated.

Yield: 218 mg (21.9%)

MS (ESIpos): m/z=740 [M+H]⁺

1H NMR (500 MHz, CHLOROFORM-d) δ ppm 0.88 (t, 9H), 0.97-1.09 (m, 6H), 1.28-1.57 (m, 18H), 1.89-1.92 (m, 2H), 2.65-2.69 (m, 1H), 2.76-2.85 (m, 2H), 4.17-4.19 (m, 1H), 4.86-4.88 (m, 1H), 7.12-7.13 (d, 2H), 7.33-7.35 (d, 2H)

### Example 10

### (2S,4S)-2-Amino-4-(4-[I-125]-iodo-benzyl)-pentanedioic acid

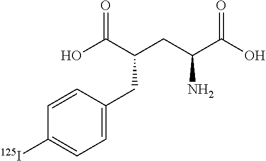 [SMILES: N[C@@H](C[C@H](Cc1ccc([125I])cc1)C(=O)O)C(=O)O]

25 μL of a solution of 0.1 N [¹²⁵I]NaI (360.6 MBq) in 0.1 N NaOH were incubated for 5 min at 25° C. together with 25 μL 0.05 N phosphoric acid (H3PO4), 500 pg of (2S,4S)-2-tert-butoxycarbonylamino4-(4-tributylstannanyl-benzyl)-entanedioic acid di-tert-butyl ester (9) in 100 μL ethanol and 25 μL chloramin-T solution (1 mg/100 μL 0.1 N K₂HPO₄). After incubation the reaction mixture diluted with 1 mL water/acetonitrile (1:1) and subsequently transferred to the HPLC unit using a remote-control-operated HPLC injection system and subjected to a semi-preparative HPLC purification using a Agilent Zorbax Bonus-RP C18, 5 μm; 250_{—}9.4 mm column. Eluent was acetonitrile/water with 0.1% trifluoroacetic acid at a flow of 4 ml/min. For the purification a linear gradient from 60 to 100% acetonitrile within 15 min was used. The collected HPLC-fraction (retention time: 17.4 min) was diluted with 15 mL water and given on a C18 plus cartridge (Waters). After washing with 10 mL water the activity was eluted with 2 mL ethanol. To this solution were added 300 μL 4 N HCl and heated for 10 min at 110° C. in an open Wheaton vial under slight nitrogen stream. The residue was diluted with 2 mL water/acetonitrile (9:1) and subsequently transferred to the HPLC unit using a remote-control-operated HPLC injection system and subjected to a semi-preparative HPLC purification using a Agilent Zorbax Bonus-RP C18, 5 μm; 250_{—}9.4 mm column. Eluent was acetonitrile/water with 0.1% trifluoroacetic acid at a flow of 4 ml/min. For the purification a linear gradient from 10 to 50% acetonitrile within 20 min was used. The collected HPLC-fraction (retention time:13.9 min) was diluted with 18 mL water and given on a C18 plus cartridge (Waters). After washing with 5 mL water for two times the activity was eluted with 1 mL ethanol to get 113.3 MBq of the final tracer in a radiochemical yield of 31% and a radiochemical purity of 99% after a synthesis time of 126 min. The specific activity of the final tracer was 42.9 GBq/μmol.

### Example 11

### (2S,5S)-2-Amino-5-(4-iodo-benzyl)-hexanedioic acid

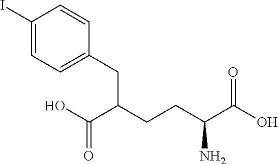 [SMILES: N[C@@H](CCC(Cc1ccc(I)cc1)C(=O)O)C(=O)O]

(11 a) (S)-2-tert-Butoxycarbonylamino-hexanedioic acid di-tert-butyl ester

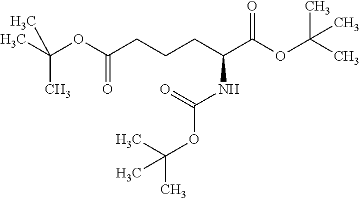 [SMILES: CC(C)(C)OC(=O)CCC[C@H](NC(=O)OC(C)(C)C)C(=O)OC(C)(C)C]

13.67 g (50 mmol) of di-tert-butyl-L-alpha-aminoadipate (J Med Chem 1994, 37(20), 3294-3302) were dissolved in 150 mL of tetrahydrofuran (THF). 20.79 mL (150 mmol) of triethylamine and a solution of 14.19 g (65 mmol) di-tert-butyl dicarbonate in 50 mL of THF were added. The mixture was stirred at room temperature overnight and the solvent was concentrated in vacuo. The residue was taken up in water and ethyl acetate, the organic phase was separated off, washed with water until neutral, dried over sodium sulphate and filtered, and the filtrate was concentrated. The crude product obtained in this manner was chromatographed on silica gel using a hexane/ethyl acetate gradient, and the appropriate fractions were combined and concentrated in vacuo.

Yield: 8.4 g (45.0%)

MS (ESIpos): m/z=374 [M+H]⁺

1H NMR (400 MHz, CHLOROFORM-d) δ ppm 1.43-1.46 (m, 27H), 1.58-1.65 (m, 3H), 1.76-1.79 ( m, 1H), 2.22-2.25 (m, 2H), 4.12-4.19 (m, 1H), 5.02-5.04 (m, 1H)

(11 b) (S)-2-Amino-5-(4-iodobenzyl)-hexanedioic acid

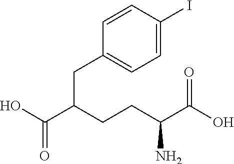 [SMILES: N[C@@H](CCC(Cc1ccc(I)cc1)C(=O)O)C(=O)O]

1.87 g (5 mmol) of (S)-2-tert-Butoxycarbonylamino-hexanedioic acid di-tert-butyl ester (11a) were dissolved in 25 mL of THF and cooled to −70° C. 11 mL (11 mmol) of a 1M solution of lithium bis(trimethylsilyl)amide in THF were added dropwise over a period of 30 min at this temperature and the mixture was stirred at −70° C. for 2 hours. 1.93 g (6.5 mmol) of 4-iodo-benzyl bromide were then added and after 3 h at this temperature, the cooling bath was removed and 25 mL of 2N aqueous hydrochloric acid and 100 mL of dichloromethane added. The organic phase was separated off, washed with water until neutral, dried over sodium sulphate and filtered, and the filtrate was concentrated. The crude product obtained in this manner was chromatographed on silica gel using a hexane/ethyl acetate gradient, and the appropriate fractions were combined and concentrated (75 mg). MS (ESIpos): m/z=590 [M+H]⁺

The residue was dissolved in 3 mL of trifluoroacetic acid and stirred overnight at room temperature. The reaction mixture was then evaporated to dryness and the resulting crude product was then chromatographed with water/methanol on C18-silica gel and the resulting fractions were combined and reduced in volume by evaporation.

Yield: 7.5 mg (0.4%)

MS (ESIpos): m/z=378 [M+H]⁺

1H NMR (600 MHz, DEUTERIUM OXIDE) δ ppm 1.36-1.48 (m, 2H), 1.63-1.76 (m, 2H), 2.33-2.40 (m, 1H), 2.56-2.63 (m, 2H), 3.51-3.61 (m, 1H), 6.89-6.92 (d, 2H), 7.53-7.57 (d, 2H)

In analogy to Example 11, (S)-2-tert-Butoxycarbonylamino-hexanedioic acid di-tert-butyl ester can be alkylated with other iodinated bromomethyl (hetero)aryl derivatives or the respective iodomethyl (hetero)aryl derivatives followed by deprotection.

### Example 12

Cell uptake & Retention of (2S,4S)-2-Amino-4-(4-[1-125]-iodo-benzyl)-pentanedioic acid—For determination of the biological activity of (2S,4S)-2-Amino-4-(4-[I-125]-iodo-benzyl)-pentanedioic acid, the I-125 labeled compound was used as tracer in a cell uptake experiment using H460 (human NSCLC) cells. Approximately 100.000 cells were incubated with 0.25 MBq (2S,4S)-2-Amino-4-(4-[I-125]-iodo-benzyl)-pentanedioic acid for up to 60 minutes in PBS-buffer containing 0.1% BSA and the cell-bound fraction was determined. A time-dependent uptake was observed during the 60 min incubation period. Approximately 22,3% of applied dose was taken up by the cells during the 60 min incubation period (see FIG. 6).

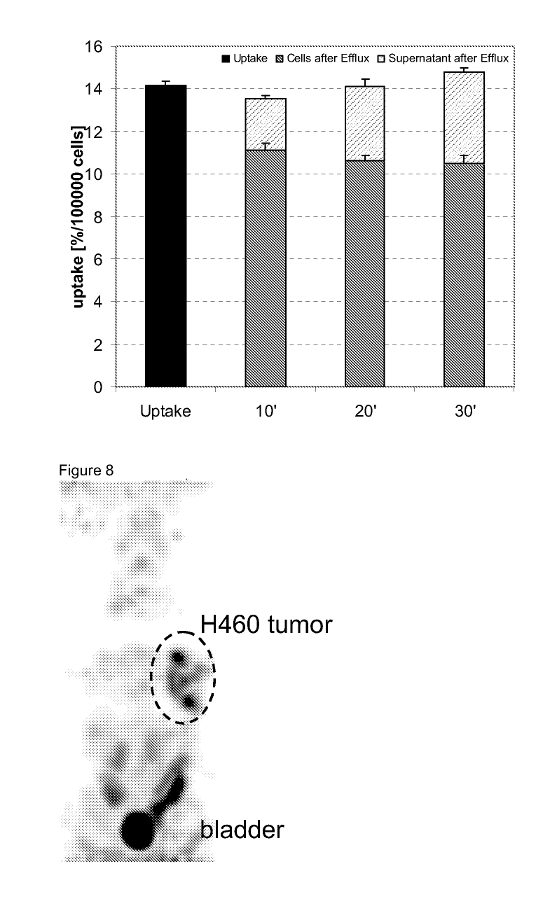

In a second experiment, the retention of activity in tumor cells was examined. H460 cells were loaded with 0.25 MBq (2S,4S)-2-Amino-4-(4-[I-125]-iodo-benzyl)-pentanedioic acid for 30 minutes in PBS/BSA-buffer. After this uptake, the buffer was removed and the cells were washed with PBS. The cells were then incubated with new PBS-buffer (without activity) for up to 30 min. The release of activity into the supernatant as well as the retention of activity inside the cells was examined. It was discovered, that more than 75% of activity were retained in the tumor cells after 30 min under these efflux conditions (see FIG. 7).

### Example 13

Biodistribution in H460 tumor bearing mice. To test the pharmacokinetic properties of (2S,4S)-2-Amino-4-(4-[I-125]-iodo-benzyl)-pentanedioic acid, the iodinated compound was examined in H460 tumor bearing mice. NMRI (nu/nu) mice were inoculated with H460 tumor cells 8 to 10 days before the biodistribution studies. 185 kBq of activity of the tracer was injected into each mouse. n=3 mice were used at every time point. After injection of the I125-labeled compound, mice were sacrificed at the time points indicated. All organs were removed and radioactivity was determined using a γ-counter. A good uptake in the tumor (4.12% injected dose per gram of tumor at 30 min p.i.) was observed. Very rapid clearance of radioactivity takes place via the kidneys, with more than 90% of activity being excreted after 30 min p.i. The biodistribution data suggest excellent SPECT imaging properties of (2S,4S)-2-Amino-4-(4-[I-125]-iodo-benzyl)-pentanedioic acid (see Table 1).

TABLE 1
Biodistribution in H460 tumor bearing mice

| | timepoint | | | | | | | |
|---|---|---|---|---|---|---|---|---|
| | 0.5 h | | 1 h | | 2 h | | 4 h | |
| | | S.D. | | S.D. | | S.D. | | S.D. |
| % Dosis/g | | | | | | | | |
| liver | 0.25 | 0.04 | 0.07 | 0.02 | 0.03 | 0.01 | 0.03 | 0.01 |
| kidney | 2.14 | 0.39 | 0.83 | 0.13 | 0.24 | 0.04 | 0.10 | 0.02 |
| tumor | 4.12 | 0.45 | 3.68 | 0.56 | 1.90 | 0.39 | 1.55 | 0.19 |
| blood | 0.28 | 0.02 | 0.13 | 0.00 | 0.08 | 0.01 | 0.06 | 0.01 |
| thyroid | 0.43 | 0.10 | 0.72 | 0.59 | 0.42 | 0.09 | 1.05 | 0.61 |
| gallbladder | 8.09 | 3.50 | 5.00 | 4.32 | 4.73 | 1.89 | 6.63 | 1.51 |
| intestine | 1.26 | 0.14 | 0.85 | 0.69 | 0.78 | 0.22 | 1.32 | 0.16 |
| pancreas | 0.62 | 0.17 | 0.12 | 0.08 | 0.06 | 0.02 | 0.03 | 0.01 |
| Bilanz/summary | | | | | | | | |
| Recovery | 112.7 | 1.4 | 109.3 | 5.7 | 109.7 | 4.7 | 115.3 | 0.9 |
| organs | 10.2 | 2.0 | 6.8 | 1.0 | 3.8 | 0.5 | 2.8 | 0.6 |
| carcass | 3.4 | 1.0 | 2.2 | 0.6 | 1.1 | 0.5 | 0.5 | 0.0 |
| urine | 99.1 | 2.9 | 99.6 | 4.6 | 102.8 | 5.5 | 111.9 | 0.5 |
| faeces | — | — | 0.7 | 1.0 | 2.0 | 2.4 | 0.1 | 0.2 |

### Example 14

SPECT imaging. (2S,4S)-2-Amino-4-(4-[I-125]-iodo-benzyl)-pentanedioic acid was examined in NCl-H460 (human NSCLC) tumor bearing nude-mice (NMRI nu/nu). Approx. 10 MBq of (2S,4S)-2-Amino-4-(4-[I-125]-iodo-benzyl)pentanedioic acid was injected into the mouse. SPECT imaging was performed using a γ-camera (Nucline SPIRIT DH-V). Images were aquired at 60 min p.i. for 35 min with 60 sec/frame. The tumor was very well visible in these SPECT-images (see FIG. 8).

### Example 15

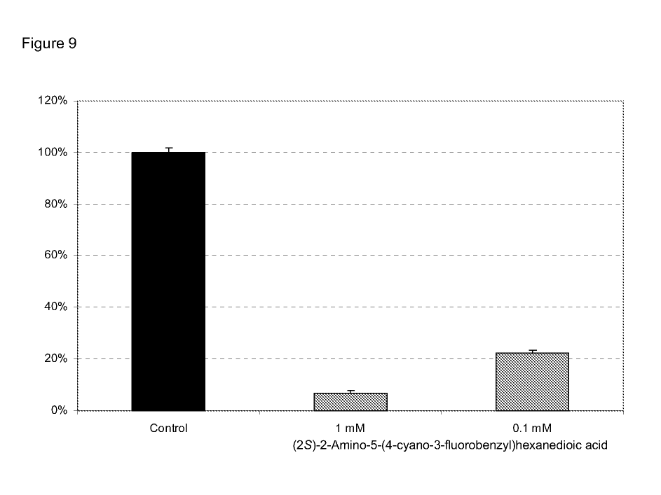

The ability of (S)-2-Amino-5-(4-iodobenzyl)-hexanedioic acid to compete with uptake of glutamic acid into tumor cells was examined. Therefore, tumor cells were co-incubated with 3H-labeled glutamic acid and (S)-2-Amino-5-(4-iodobenzyl)-hexanedioic acid. This compounds was used in large excess to the tracer 3H-glutamic acid. Two concentrations were examined (1mM an 0.1 mM). Surprisingly, this compound strongly reduces the uptake of glutamic acid, indicating that the same transport systems may be exploited by the test-compounds. See FIG. 9.

1. A compound of the general formula (I) 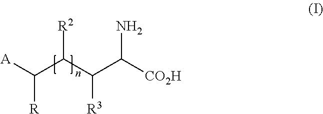
wherein
n=0 or 1;
A is selected from the group comprising 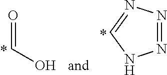
wherein * indicates the atom of connection of A;
R² and R³ are Hydrogen,
R¹ is X
wherein X is
Iodo-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
Iodo-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S. and wherein the heteroaryl moiety is optionally substituted by a methyl group or
Iodo-CH═CH—(CH₂)ₘ, wherein m=1-3 and
encompassing single isomers, diastereomers, tautomers, E- and Z-isomers, enantiomers, mixtures thereof, and suitable salts thereof.

2. The compound according to claim 1 selected from
(2S,4S)-2-Amino-4-(4-hydroxy-3-iodo-benzyl)-pentanedioic acid 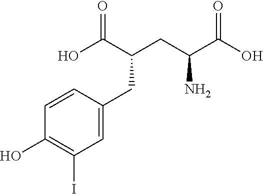 [SMILES: N[C@@H](C[C@H](Cc1ccc(O)c(I)c1)C(=O)O)C(=O)O]
(2S,4S)-2-Amino-4-(4-hydroxy-3-[125-I]iodo-benzyl)-pentanedioic acid 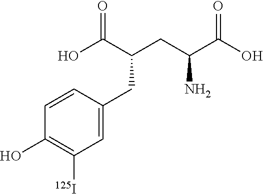 [SMILES: N[C@@H](C[C@H](Cc1ccc(O)c([125I])c1)C(=O)O)C(=O)O]
(2S,4S)-2-Amino-4-[3-(4-iodo-phenoxy)-propyl]-pentanedioic acid 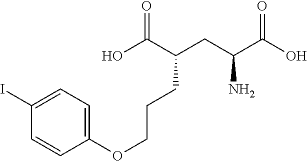 [SMILES: N[C@@H](C[C@H](CCCOc1ccc(I)cc1)C(=O)O)C(=O)O]
(2S,4S)-2-Amino-4-3-(4-[125-I]iodo-phenoxy)-propyl]-pentanedioic acid 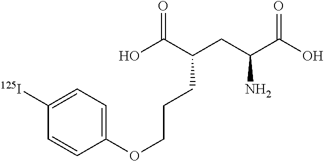 [SMILES: N[C@@H](C[C@H](CCCOc1ccc([125I])cc1)C(=O)O)C(=O)O]
(2S,4S)-2-Amino-4-(4-iodo-benzyl)-pentanedioic acid 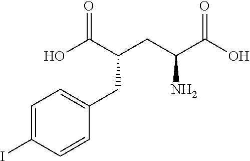 [SMILES: N[C@@H](C[C@H](Cc1ccc(I)cc1)C(=O)O)C(=O)O]
(2S,5S)-2-Amino-5-(4-iodo-benzyl)-hexanedioic acid 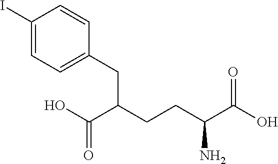 [SMILES: N[C@@H](CCC(Cc1ccc(I)cc1)C(=O)O)C(=O)O]
and
(2S,4S)-2-Amino-4-(4[125-I]iodo-benzyl)-pentanedioic acid

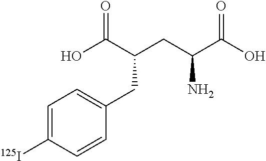 [SMILES: N[C@@H](C[C@H](Cc1ccc([125I])cc1)C(=O)O)C(=O)O]

3. A compound of the general formula (II) 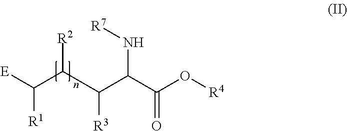
wherein
n=0 or 1;
E is selected from the group comprising 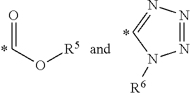
wherein * indicates the atom of connection of E;
R² and R³ are Hydrogen,
R¹ is X,
wherein X is
Iodo-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
Iodo-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S and wherein the heteroaryl moiety is optionally substituted by a methyl group or
Iodo-CH═CH—(CH₂)ₘ, wherein m=1-3;
R⁴=Hydrogen or O-protecting group;
R⁵=Hydrogen or O-protecting group;
R⁶=Hydrogen or triphenylmethyl;
R⁷=Hydrogen or N-protecting group;
with the proviso, that at least one of the substituents R⁴, R⁵, R⁶ or R⁷ is not Hydrogen and encompassing single isomers, diastereomers, tautomers, E- and Z-isomers, enantiomers, mixtures thereof, and suitable salts thereof.

4. The compound according to claim 3 selected from
(2S,4S)-2-tert-Butoxycarbonylamino-4-[3-(4-iodo-phenoxy)-propyl]-pentanedioic acid di-tert-butvl ester 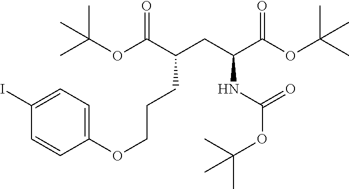 [SMILES: CC(C)(C)OC(=O)N[C@@H](C[C@H](CCCOc1ccc(I)cc1)C(=O)OC(C)(C)C)C(=O)OC(C)(C)C]
and
(2S,4S)-2-tert-Butoxycarbonylamino-4-(4-[125-I]iodo-benzyl)-pentanedioic acid di-tert-butyl ester

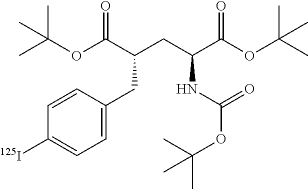 [SMILES: CC(C)(C)OC(=O)N[C@@H](C[C@H](Cc1ccc([125I])cc1)C(=O)OC(C)(C)C)C(=O)OC(C)(C)C]

5. A compound of the general formula (III) 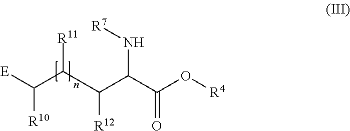
wherein
n=0 or 1;
E is selected from the group comprising 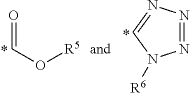
wherein * indicates the atom of connection of E;
R¹¹ and R¹² are Hydrogen,
R10 is Y,
wherein Y is
L-aryl-G-CH₂, wherein G is a direct bond or C₁-C₅ alkyl wherein a methylene group of the alkyl chain may optionally be replaced by an oxygen atom or by a nitrogen atom and wherein a methylene group may be substituted with an oxo group (═O) and wherein the aryl moiety is optionally substituted by 1 or 2 substituents independently selected from R⁹, OH, OR⁹, NH₂, NHR⁹, NR⁹R⁹
wherein R⁹ is C₁-C₃-alkyl, preferably methyl;
L-heteroaryl-G-CH₂, wherein G is a direct bond or C₁-C₅ alkyl, wherein a methylene group of the alkyl chain may optionally be replaced by an oxygen atom or by a nitrogen atom and wherein a methylene group may be substituted with an oxo group (═O) and wherein heteroaryl comprises 5 to 6 ring atoms wherein 1 or 2 atoms are independently selected from N, O or S and wherein the heteroaryl moiety is optionally substituted by a methyl group or
L-CH═CH—(CH₂)ₘ, wherein m=1-3
wherein L is
(R¹³)₃Sn, (R¹³)₃Si or (HO)₂B,
wherein R¹³ is C₁-C₄ Alkyl, preferably n-Butyl;
R⁴=Hydrogen or O-protecting group;
R⁵=Hydrogen or O-protecting group;
R⁶=Hydrogen or triphenylmethyl;
R⁷=Hydrogen or N-protecting group and encompassing single isomers, diastereomers, tautomers, E- and Z-isomers, enantiomers, mixtures thereof, and suitable salts thereof.

6. The compound according to claim 5 selected from
(2S,4S)-2-tert-Butoxycarbonylamino-4-(4-tributylstannanyl-benzyl)-pentanedioic acid di-tert-butyl ester

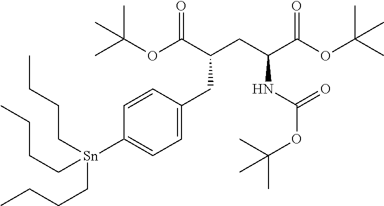 [SMILES: CCC[CH2][Sn]([CH2]CCC)([CH2]CCC)[c]1ccc(C[C@@H](C[C@H](NC(=O)OC(C)(C)C)C(=O)OC(C)(C)C)C(=O)OC(C)(C)C)cc1]

7. A composition comprising compounds of the general formula (I), (II), (III), or mixture thereof according to claim 1 and pharmaceutically acceptable carrier or diluent.

8. A method for obtaining compounds of formula (I), (II) or mixtures thereof according to claim 1 comprising the steps
Reacting a compound of general formula (III) with an Iodine containing moiety wherein the Iodine is ¹²³I, ¹²⁴I, ¹²⁵I, ¹²⁷I, or ¹³¹I,
Optionally deprotecting compound of formula (II) and
Optionally converting obtained compound into a suitable salt of inorganic or organic acids thereof, hydrates, complexes and solvates thereof.

9. A compound of general formula (I) or (II) or mixtures thereof according to claim 1 for the manufacture of an imaging tracer for imaging proliferative diseases.

10. A kit comprising a sealed vial containing a predetermined quantity of a compound having general chemical Formula (I), (II) or (III) or mixtures thereof according to claim 1 and suitable salts of inorganic or organic acids thereof, hydrates, complexes, esters, amides, and solvates thereof.

11. A compound of general formula (I) or (II) or mixtures thereof according to claim 1 for the manufacture of a medicament for radiotherapy of proliferative diseases wherein the iodine isotope is ¹³¹I.
